(19) United States
(12) Patent Application Publication
(10) Pub. No.: US 2025/0011484 A1
(43) Pub. Date: Jan. 9, 2025

(54) METHODS FOR PREPARING METALLOCENE COMPOUNDS

(71) Applicant: Chevron Phillips Chemical Company LP, The Woodlands, TX (US)

(72) Inventors: Graham R. LIEF, Bartlesville, OK (US); Qing YANG, Bartlesville, OK (US); Eric HASCHKE, Bartlesville, OK (US)

(21) Appl. No.: 18/751,434

(22) Filed: Jun. 24, 2024

Related U.S. Application Data
(60) Provisional application No. 63/510,495, filed on Jun. 27, 2023.

(51) Int. Cl. C08F 210/16; C07C 2/30; C07F 17/00

(52) U.S. Cl. CPC C08F 210/16; C07C 2/30; C07F 17/00; C07C 2531/12

(57) ABSTRACT

Disclosed herein are synthetic methods for improving the solubility of metallocenes such as those that contain perfluorobenzyl(indene) groups. These methods can include alkylation of the perfluoroaryl ring of metallocene precursors using alkyl lithium reagents in a one-pot reaction. Further, these methods can be employed without altering the equipment or conditions of the reaction, without isolation of new intermediates, and while generally retaining desired catalyst characteristics.

### REFERENCE TO RELATED APPLICATION

This application claims the benefit of U.S. Provisional Patent Application No. 63/510,495, filed on Jun. 27, 2023, the disclosure of which is incorporated herein by reference in its entirety.

### FIELD OF THE INVENTION

The present disclosure generally relates to methods for preparing metallocene compounds and the use of the metallocene compounds within catalyst compositions for oligomerization and polymerization processes, and more particularly relates to preparing metallocene compounds with improved solubility.

### BACKGROUND

Metallocene compounds have been developed as effective catalysts for oligomerization and polymerization processes. Structural properties of metallocenes have been finely tuned to produce desired oligomer and polymer characteristics. For example, metallocene compounds comprising at least one indenyl ligand containing at least one halogenated substituent can produce polyethylene having low levels of short chain branching. There is a need for metallocene compounds with improved solubility without altering the properties of the oligomer and polymer produced from a given metallocene compound. Accordingly, it is to this end that the present invention is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Methods for the preparation of a metallocene compound are disclosed herein, comprising (i) contacting a first compound having formula Cp^{A}-(CH₂)ₙ—Ar—X with a Bronsted base to form a deprotonated compound; (ii) contacting the deprotonated compound with a substitution reagent to form a substituted compound having formula Cp^{A}-(CH₂)ₙ—Ar—R^{x}; and (iii) contacting the substituted compound with a second compound having formula Cp^{B}-M-X₃ to form a metallocene compound having formula (I):

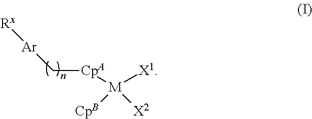

In certain aspects, M can be Zr, Ti, or Hf; each X independently can be a halogen or NR^{y}₂; X¹ and X² each can be a monoanionic ligand; Cp^{A} can be a cyclopentadienyl, indenyl, or fluorenyl group, optionally substituted with one or more other substituents; Cp^{B} can be a substituted or unsubstituted cyclopentadienyl, indenyl, or fluorenyl group; Ar can be an aryl group comprising a halogen substituent; R^{x} can be a C₁ to C₁₈ hydrocarbyl group substituent on Ar (e.g., selected from alkyl or alkenyl or aryl; a phenyl group, a benzyl group, a C₁ to C₈ alkyl group, or a C₃ to C₈ alkenyl group); and n can be an integer from 0 to 5.

Metallocene compounds are also disclosed herein and can have formula (I) and substituents generally as referenced above. Metallocene compounds disclosed herein can have an improved solubility, relative to metallocenes without an R^{x}-substituent. Catalyst compositions are also disclosed herein and can comprise metallocene compounds as described above, an activator, and an optional co-catalyst. In certain aspects, the activator can comprise an aluminoxane compound, an organoboron or organoborate compound, an ionizing ionic compound, a chemically treated solid oxide, or any combination thereof.

Oligomerization processes are disclosed herein and can comprise contacting a catalyst composition with an alpha olefin monomer, and optionally H₂, under oligomerization conditions to produce an oligomer product. Polymerization processes are disclosed herein and can comprise contacting a catalyst composition with an ethylene monomer and an optional α-olefin comonomer in a polymerization reactor system under polymerization conditions to produce an ethylene polymer.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects and embodiments may be directed to various feature combinations and sub-combinations described in the detailed description.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and/or feature disclosed herein, all combinations that do not detrimentally affect the compounds, compositions, and/or methods described herein are contemplated with or without explicit description of the particular combination. Additionally, unless explicitly recited otherwise, any aspect and/or feature disclosed herein can be combined to describe inventive features consistent with the present disclosure.

While compositions and methods are described herein in terms of “comprising” various components or steps, the compositions and methods also can “consist essentially of” or “consist of” the various components or steps, unless stated otherwise. For example, a catalyst composition consistent with aspects of the present invention can comprise; alternatively, can consist essentially of; or alternatively, can consist of; a metallocene compound, a co-catalyst, and a chemically treated solid oxide.

The terms “a,” “an,” “the,” etc., are intended to include plural alternatives, e.g., at least one, unless otherwise specified. For instance, the disclosure of “a co-catalyst” or “a metallocene compound” is meant to encompass one, or mixtures or combinations of more than one, co-catalyst or metallocene compound, respectively, unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any particular compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise; e.g., a general reference to pentane includes n-pentane, 2-methyl-butane, and 2,2-dimethylpropane, while a general reference to a butyl group includes an n-butyl group, a sec-butyl group, an iso-butyl group, and a tertbutyl group. Additionally, the reference to a general structure or name encompasses all enantiomers, diastereomers, and other optical isomers whether in enantiomeric or racemic forms, as well as mixtures of stereoisomers, as the context permits or requires.

For any particular formula or name that is presented, any general formula or name presented also encompasses all conformational isomers, regioisomers, and stereoisomers that can arise from a particular set of substituents.

The term “metallocene” as used herein describes compounds comprising at least one η³ to η⁵-cycloalkadienyl-type moiety, wherein η³ to η⁵-cycloalkadienyl moieties include cyclopentadienyl ligands, indenyl ligands, fluorenyl ligands, and the like, including partially saturated or substituted derivatives or analogs of any of these. Possible substituents on these ligands can include H, therefore this invention comprises ligands such as tetrahydroindenyl, tetrahydrofluorenyl, octahydrofluorenyl, partially saturated indenyl, partially saturated fluorenyl, substituted partially saturated indenyl, substituted partially saturated fluorenyl, and the like. In some contexts, the metallocene is referred to simply as the “catalyst,” in much the same way the term “co-catalyst” is used herein to refer to, for example, an organoaluminum compound.

The term “hydrocarbon” refers to a compound containing only carbon and hydrogen. Other identifiers can be utilized to indicate the presence of particular groups in the hydrocarbon (e.g., halogenated hydrocarbon indicates the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “co-catalyst” is used generally herein to refer to compounds such as aluminoxane compounds, organoboron or organoborate compounds, ionizing ionic compounds, organoaluminum compounds, organozinc compounds, organomagnesium compounds, organolithium compounds, and the like, that can constitute one component of a catalyst composition, when used, for example, in addition to a chemically treated solid oxide activator. The term “co-catalyst” is used regardless of the actual function of the compound or any chemical mechanism by which the compound may operate.

The term “substituted” when used to describe a group, for example, when referring to a substituted analog of a particular group, is intended to describe any non-hydrogen moiety that formally replaces a hydrogen in that group, and is intended to be non-limiting. A group or groups can also be referred to herein as “unsubstituted” or by equivalent terms such as “non-substituted,” which refers to the original group in which a non-hydrogen moiety does not replace a hydrogen within that group. Unless otherwise specified, “substituted” is intended to be non-limiting and include inorganic substituents or organic substituents as understood by one of ordinary skill in the art.

The term “olefin” refers to hydrocarbons that have at least one carbon-carbon double bond that is not part of an aromatic ring or an aromatic ring system. The term “olefin” includes aliphatic and aromatic, cyclic and acyclic, and/or linear and branched hydrocarbons having at least one carbon-carbon double bond that is not part of an aromatic ring or ring system unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s).

The term “alpha olefin” as used herein refers to any olefin that has 1) a carbon-carbon double bond between the first and second carbon atom of the longest contiguous chain of carbon atoms, and 2) at least one hydrogen atom bound to the second carbon of the chain. The term “alpha olefin” includes linear and branched alpha olefins and alpha olefins which can have more than one non-aromatic carbon-carbon double bond, unless expressly stated otherwise. In the case of branched olefins, a branch can be at the 2-position of a 1-alkene (a vinylidene) with respect to the olefin double bond.

The term “polymer” is used herein generically to include olefin homopolymers, copolymers, terpolymers, and the like, as well as alloys and blends thereof. The term “polymer” also includes impact, block, graft, random, and alternating copolymers. A copolymer is derived from an olefin monomer and one olefin comonomer, while a terpolymer is derived from an olefin monomer and two olefin comonomers. Accordingly, “polymer” encompasses copolymers and terpolymers derived from any olefin monomer and comonomer(s) disclosed herein. Similarly, the scope of the term “polymerization” includes homopolymerization, copolymerization, and terpolymerization. Therefore, an ethylene polymer includes ethylene homopolymers, ethylene copolymers (e.g., ethylene/α-olefin copolymers), ethylene terpolymers, and the like, as well as blends or mixtures thereof. Thus, an ethylene polymer encompasses polymers often referred to in the art as LLDPE (linear low density polyethylene) and HDPE (high density polyethylene). As an example, an olefin copolymer, such as an ethylene copolymer, can be derived from ethylene and a comonomer, such as 1-butene, 1-hexene, or 1-octene. If the monomer and comonomer were ethylene and 1-hexene, respectively, the resulting polymer can be categorized an as ethylene/1-hexene copolymer. The term “polymer” also includes all possible geometrical configurations, unless stated otherwise, and such configurations can include isotactic, syndiotactic, and random symmetries. Moreover, unless stated otherwise, the term “polymer” also is meant to include all molecular weight polymers.

The terms “catalyst composition,” “catalyst mixture,” “catalyst system,” and the like, do not depend upon the actual product or composition resulting from the contact or reaction of the initial components of the disclosed or claimed catalyst composition/mixture/system, the nature of the active catalytic site, or the fate of the co-catalyst, metallocene compound, or activator, after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, can be used interchangeably throughout this disclosure.

The terms “contacting” and “combining” are used herein to describe compositions, processes, and methods in which the materials or components are contacted or combined together in any order, in any manner, and for any length of time, unless otherwise specified. For example, the materials or components can be blended, mixed, slurried, dissolved, reacted, treated, compounded, or otherwise contacted or combined in some other manner or by any suitable method or technique.

Several types of ranges are disclosed herein. When a range of any type is disclosed or claimed, the intent is to disclose or claim individually each possible number that such a range could reasonably encompass, including end points of the range as well as any sub-ranges and combinations of sub-ranges encompassed therein. For example, the weight ratio of the metallocene compound to the chemically treated solid oxide in the catalyst composition can be in various ranges. By a disclosure that the weight ratio of the metallocene compound to the chemically treated solid oxide can range from 1:10 to 1:10,000, the intent is to recite that the weight ratio can be any ratio within the range and, for example, can include any range or combination of ranges from 1:10 to 1:10,000, such as from 1:10 to 1:1,000, from 1:10 to 500:1, or from 1:10 to 1:100, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods, devices, and materials similar or equivalent to those described herein can be used in the practice or testing of the invention, the typical methods, devices, and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION

Disclosed herein are methods for preparing metallocene compounds that have hydrophobic substituents for improved solubility. These methods can be conducted using a one-pot procedure, and therefore in certain aspects may exclude the isolation of air- and moisture-sensitive intermediates. Metallocene compounds prepared from these methods also are disclosed herein. Surprisingly, these metallocene compounds demonstrate improved solubility while retaining comparable catalytic performance in both oligomerization processes and polymerization processes.

### Methods for Preparing Metallocene Compounds

Conventional metallocene preparations require isolation of a cyclopentadienyl-containing intermediate prior to coordination to a metal compound, and therefore novel preparatory syntheses can be required for each new metallocene compound desired. Metallocene syntheses disclosed herein involve the modification of known cyclopentadienyl-containing metallocene substrates in situ, and within the reaction framework (e.g., reaction systems, conditions) employed by conventional metallocene synthesis schemes. The disclosed methods for preparing metallocene compounds can be applied to any metallocene compounds suitable for use within a catalyst composition and in which improved solubility may be beneficial.

In certain aspects, methods for the preparation of a metallocene compound can comprise (i) contacting a first compound having a formula Cp^{A}-(CH₂)ₙ—Ar—X with a Bronsted base to form a deprotonated compound having a formula Cp^{A}(−)—(CH₂)ₙ—Ar—X; (ii) contacting the deprotonated compound with a substitution reagent to form a substituted compound having formula Cp^{A}-(CH₂)ₙ—Ar—R^{x}; and (iii) contacting the substituted compound with a second compound having formula Cp^{B}-M-X₃ to form a metallocene compound having formula (I):

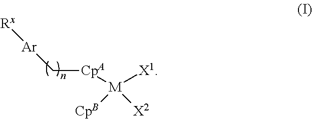

Generally, as represented in formula (I) and defined further below, each M can be Zr, Ti, or Hf; X can be a halogen or NR^{y}₂; X¹ and X² each independently can be a monoanionic ligand; Cp^{A} can be a cyclopentadienyl, indenyl, or fluorenyl group, optionally substituted with one or more other substituents; Cp^{B} can be a substituted or unsubstituted cyclopentadienyl, indenyl, or fluorenyl group; Ar can be an aryl group comprising a halogen substituent; R^{x} can be a C₁ to C₁₈ hydrocarbyl group substituent on Ar (e.g., selected from alkyl or alkenyl or aryl; a phenyl group, a benzyl group, a C₁ to C₈ alkyl group, or a C₃ to C₈ alkenyl group); and n can be an integer from 0 to 5. However, it will be understood that the methods for preparing metallocene compounds disclosed herein can be applied to any metallocene as described hereinbelow. Moreover, it follows that the first compound and second compounds can be any combination of formula components listed above suitable for preparation of any metallocene compound.

Processes disclosed herein can comprise deprotonating a first compound comprising a cyclopentadienyl moiety (Cp^{A} as described above) by contacting the first compound with a Bronsted base. In the first compound, X can be a halogen or NR^{y}; alternatively, X can be Cl; alternatively, X can be Br; alternatively, X can be F. In certain aspects, each R^{y} independently can be a C₁ to C₈ hydrocarbyl group, e.g., a methyl group, an ethyl group, a propyl group, a butyl group, a pentyl group, a hexyl group, a heptyl group, or an octyl group; alternatively, a methyl group, an ethyl group, a butyl group, a hexyl group, an octyl group; alternatively, a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

In the first compound (Cp-(CH₂)ₙ—Ar—X), Ar also can be further substituted, for instance by any additional number of halogen substituents. In certain aspects, the Ar group of Cp-(CH₂)ₙ—Ar—X as described above can have one or more further substituents beyond those present in the formula (e.g., one halogen substituent, or two halogen substituents, or three halogen substituents, or four halogen substituents). In certain aspects, each halogen substituent can be F. Thus, Ar group of the first compound can comprise one F, or two F, or three F, or four F, or 5 F, arranged at any position of Ar. In certain aspects, the first compound can comprise Ar as a 2,6-difluoroaryl group, a 2,4,6-trifluoroaryl group, a 2,3,4,5,6-pentafluoryl group, a 4-fluoroaryl group, and so forth. Suitable Bronsted bases generally can be defined as, and include, any compound that is capable of accepting a proton from the first compound. Thus, in the context of embodiments contemplated herein, the Bronsted base can be any species or compound capable of accepting or abstracting a proton from the cyclopentadienyl moiety of the first compound (Cp^{A}). While not being bound by theory, given that cyclopentadiene has a pKa of about 15, in certain aspects suitable Bronsted bases generally can include those having a conjugate acid with a pKa of about 15 or above. In certain aspects, the Bronsted base can be a metal carbonate, a metal acetylide, a t-butoxide salt, an enolate, a metal hydride, a metal amide, an organolithium or an organomagnesium halide, and the like. Of these, organolithiums and organomagnesium halides may be most practically applied to the preparation of certain metallocene compounds, given their hydrophobicity and solubility in organic solvents.

In certain aspects, the Bronsted base can comprise an organolithium compound or organomagnesium compound selected from methyllithium ethyllithium, n-butyl lithium, t-butyllithium, n-hexyllithium, benzyllithium, phenyllithium, methylmagnesium bromide, methylmagnesium chloride, ethylmagnesium bromide, isopropylmagnesium chloride, t-butyl magnesium chloride, vinylmagnesium bromide, allylmagnesium bromide, ethynylmagnesium chloride, phenylmagnesium chloride, benzylmagnesium chloride, and combinations thereof. In certain aspects, the Bronsted base can comprise methyllithium, n-butyllithium, t-butyllithium, n-hexyllithium, phenyllithium, and/or benzyllithium. In other aspects, the Bronsted base can comprise methyllithium, n-butyllithium, and/or n-hexyllithium.

Processes disclosed herein can further comprise contacting the deprotonated compound with a substitution reagent to form a substituted compound having the formula Cp^{A}-(CH₂)ₙ—Ar—R^{x}. The substitution reagent can be any reagent sufficiently reactive (e.g., sufficiently nucleophilic) to displace X with an R^{x} group as defined herein. In certain aspects, the substitution reagent can comprise an organolithium compound or an organomagnesium halide. Thus, as for Bronsted bases listed above, in certain aspects the substitution reagent can comprise methyllithium ethyllithium, n-butyl lithium, t-butyllithium, n-hexyllithium, benzyllithium, phenyllithium, methylmagnesium bromide, methylmagnesium chloride, ethylmagnesium bromide, isopropylmagnesium chloride, t-butyl magnesium chloride, vinylmagnesium bromide, allylmagnesium bromide, ethynylmagnesium chloride, phenylmagnesium chloride, benzylmagnesium chloride, and the like, as well as combinations thereof.

In certain aspects, the substitution reagent can be the same as the Bronsted base. Additionally, or alternatively, the Bronsted base and the substitution reagent each can comprise an organolithium reagent. In certain aspects, each of the Bronsted base and the substitution reagent can comprise methyllithium, n-butyllithium, n-hexyllithium, or combinations thereof.

Deprotonation and substitution reactions may be performed in situ, without requiring that the deprotonated compound be isolated as an intermediate prior to contacting with a substitution reagent. Thus, in certain aspects, contacting the first compound with a Bronsted base and contacting the deprotonated compound with a substitution reagent can occur in a concerted manner, by addition of two equivalents of an organolithium and/or organomagnesium halide within a one-pot synthesis. In such aspects, it will be understood that the organolithium and/or organomagnesium halide constitutes both the Bronsted base and the substitution reagent.

In any matter, the molar ratio of the Bronsted base to the first compound, can be in a range from 0.9:1 to 1.3:1. The molar ratio of the substitution reagent to the first compound also can be in a range from 0.9:1 to 1.3:1, in aspects where mono-substitution of the first compound is desired. Where multiple substitutions of the first compound are desired, the ratio of the substitution reagent to the first compound can approximate the appropriate multiple. For instance, where a di-substitution is desired, the molar ratio of the substitution reagent to the first compound can be in a range from 1.7:1 to 2.3:1. Alternatively, where a tri-substitution of the first compound is desired, the molar ratio of the substitution reagent to the first compound can be in a range from 2.7:1 to 3.3:1. Of course, when the Bronsted base and the substitution reagent are the same, the respective suitable ratios of the reagent to the first compound can be obtained by summing the typical ratios for each compound. For instance, an organolithium reagent may be used as both Bronsted base and substitution reagent such that a molar ratio of organolithium reagent to the first compound can be in a range from 1.9:1 to 2.6:1, in certain aspects, such as where monosubstitution of the first compound is desired.

Without being bound by theory, where the Bronsted base and substitution reagent are added simultaneously (e.g., when the Bronsted base and substitution reagent are added as two equivalents of an organolithium reagent) the relative reaction rate of the deprotonation and substitution reactions as described may allow the deprotonation to proceed prior to the substitution, giving a particular substituted product in high yield. Still, it will be understood that the deprotonation and substitution steps may be conducted in any order, or simultaneously.

The conditions of the substitution and deprotonation steps can be the same or different. In certain aspects, a deprotection temperature can be in a range from −78° C. to 25° C., from −40° C. to 0° C., or from −30° C. to −10° C. Similarly, in other aspects, the substitution temperature also can be in a range from −78° C. to 25° C., from −40° C. to 0° C., or from −30° C. to −10° C. The reaction temperature may be static or dynamic over the course of the reaction, within any range disclosed herein. For instance, the deprotonation may begin at −20° C. and allowed to warm to 25° C. over the course of the reaction.

The deprotonation and substitution steps may proceed for any amount of time necessary for the reaction to sufficiently complete. In certain aspects, the reaction time for either or both of the deprotonation and substitution steps may be in a range from 1 min to 1 day, or from 12 to 24 hours.

### Metallocene Compounds

Metallocene compounds prepared according to the methods above may be any that may be produced by the organo-substitution of an aryl leaving group (e.g., an aryl halide). Generally, the metallocene compounds disclosed herein can be substitution products of known metallocene compounds with an established utility in catalyst compositions. More specifically, in certain aspects the substituent can be observed on an aryl ring of the cyclopentadienyl-containing ligand, e.g., an indenyl ligand containing at least one halogenated substituent, such that a leaving group is replaced by a hydrophobic organic substituent. In this manner the metallocene compounds disclosed herein may generally have an improved solubility in hydrophobic solvents. Surprisingly, in certain aspects the organo-substitution in this region of the metallocene compound does not appreciably detract from the catalytic properties of the metallocene, generally resulting in similar catalytic activity, and similar product characteristics in oligomerization and polymerization processes (e.g., oligomer distribution, polymer molecular weight distribution).

As generally stated above, metallocene compounds disclosed herein can have Formula (I):

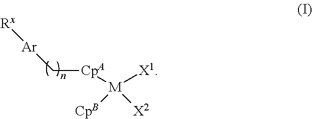

Within formula (I), M, Cp^{A}, Cp^{B}, Ar, R^{x}, X¹ and X² each are independent elements of the unbridged metallocene compound. Accordingly, the unbridged metallocene compound having formula (I) can be described using any combination of M, Cp^{A}, Cp^{B}, Ar, R^{x}, X¹ and X² disclosed herein. Unless otherwise specified, formula (I) above, any other structural formulas disclosed herein, and any metallocene complex, compound, or species disclosed herein are not designed to show stereochemistry or isomeric positioning of the different moieties (e.g., these formulas are not intended to display cis or trans isomers, or R or S diastereoisomers), although such compounds are contemplated and encompassed by these formulas and/or structures.

In accordance with aspects of this invention, the metal in formula (I), M, can be Zr, Ti, or Hf. Thus, M can be Zr in one aspect, M can be Ti in another aspect, and M can be Hf in yet another aspect.

X¹ and X² each independently can be a monoanionic ligand. In some aspects, suitable monoanionic ligands can include, but are not limited to, H (hydride), BH₄, a halide, a C₁ to C₃₆ hydrocarbyl group, a C₁ to C₃₆ hydrocarboxy group, a C₁ to C₃₆ hydrocarbylaminyl group, a C₁ to C₃₆ hydrocarbylsilyl group, a C₁ to C₃₆ hydrocarbylaminylsilyl group, —OBR¹₂, or —OSO₂R¹, wherein R¹ is a C₁ to C₃₆ hydrocarbyl group. It is contemplated that X¹ and X² can be either the same or a different monoanionic ligand. Suitable hydrocarbyl groups, hydrocarboxy groups, hydrocarbylaminyl groups, hydrocarbylsilyl groups, and hydrocarbylaminylsilyl groups are disclosed, for example, in U.S. Pat. No. 9,758,600.

In formula (I), Cp^{A} can be a cyclopentadienyl, indenyl, or fluorenyl group; either substituted as shown or further substituted, wherein each further substituent can be H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group. Importantly, each substituent on Cp^{A} can be either the same or a different substituent group. Moreover, each substituent can be at any position on the respective cyclopentadienyl, indenyl, or fluorenyl ring structure that conforms with the rules of chemical valence. In an aspect, the number of substituents on Cp^{A} and/or the positions of each substituent on Cp^{A} are independent of each other. For instance, two or more substituents on Cp^{A} can be different, or alternatively, each substituent on Cp^{A} can be the same. In these and other aspects, each substituent can be at any position on the respective cyclopentadienyl, indenyl, or fluorenyl ring structure. Therefore, Cp^{A} can have one substituent, or two substituents, or three substituents, or four substituents, or five substituents, and so forth. In certain aspects, Cp^{A} can have a C₁-C₁₂ alkyl, C₂-C₁₂ alkenyl, C₆-C₁₀ aryl, or C₇-C₁₂ aralkyl substituent (e.g., benzyl).

In formula (I), Cp^{B} can be a substituted or unsubstituted cyclopentadienyl, indenyl, or fluorenyl group. In one aspect, Cp^{A} and Cp^{B} independently can be an unsubstituted cyclopentadienyl or indenyl group. Alternatively, Cp^{A} and Cp^{B} independently can be a substituted indenyl or cyclopentadienyl group, for example, having up to 5 substituents. Cp^{A} and Cp^{B} can be the same or different. In certain aspects, Cp^{A} can be an indenyl group and Cp^{B} can be a cyclopentadienyl group.

If present, each substituent on Cp^{B} independently can be H, a halide, a C₁ to C₃₆ hydrocarbyl group, a C₁ to C₃₆ halogenated hydrocarbyl group, a C₁ to C₃₆ hydrocarboxy group, or a C₁ to C₃₆ hydrocarbylsilyl group. As above for Cp^{A}, each substituent on Cp^{B} can be either the same or a different substituent group. Moreover, each substituent can be at any position on the respective cyclopentadienyl, indenyl, or fluorenyl ring structure that conforms with the rules of chemical valence. In an aspect, the number of substituents on Cp^{B} and/or the positions of each substituent on Cp^{B} are independent of each other. For instance, two or more substituents on Cp^{B} can be different, or alternatively, all substituents on Cp^{B} can be the same. In another aspect, one or more of the substituents on Cp^{A} can be different from the one or more of the substituents on Cp^{B}, or alternatively, all substituents on both Cp^{A} and/or on Cp^{B} can be the same. In these and other aspects, each substituent can be at any position on the respective cyclopentadienyl, indenyl, or fluorenyl ring structure. If substituted, Cp^{B} independently can have one substituent, or two substituents, or three substituents, or four substituents, or five substituents and so forth. As above for Cp^{A}, in certain aspects, Cp^{B} can have a C₁-C₁₂ alkyl, C₂-C₁₂ alkenyl, C₆-C₁₀ aryl, or C₇-C₁₂ aralkyl substituent (e.g., benzyl). Cp^{B} also can be substituted similarly to Cp^{A} as represented in Formula (I). Thus, in certain aspects, Cp^{B} can have the substituent —(CH₂)ₙArR^{x}. Alternatively, Cp^{B} can be unsubstituted.

In certain aspects, Ar can be a hydrocarbon aryl group. In an aspect, the aromatic ring, Ar, of the metallocene compound can be a C₅ to C₃₀ aromatic group, or alternatively, a C₅ to C₂₀ aromatic group. In other aspects, Ar can be a heteroaryl group, for example, pyrrolidinyl, pyrrolinyl, furanyl, thiopheneyl, imidazoleyl, oxazoleyl, thiazoleyl, indoleyl, pyridineyl, pyrazineyl, isoxazoleyl, pyrazoleyl, pyrroleyl, isothiazoleyl, oxadiazoleyl, triazoleyl, indoleyl, carbazoleyl, benzofuranyl, or benzothiopheneyl.

In aspects disclosed herein, Ar can be substituted at least by R^{x}, which generally can represent the aryl organosubstituent added by substitution reagent in processes disclosed above. Thus, in certain aspects, R^{x} can be any nucleophilic moiety within the substitution reagents disclosed above. For example, where the substitution reagent is methyllithium, R^{x} can be a methyl group. In aspects where the substitution reagent is phenylmagnesium chloride, R^{x} can be a phenyl group. In other aspects, R^{x} can be a C₁ to C₁₈ hydrocarbyl group substituent (e.g., selected from alkyl or alkenyl or aryl; a phenyl group, a benzyl group, a C₁ to C₈ alkyl group, or a C₃ to C₈ alkenyl group). In still further aspects, R^{x} can be selected from methyl, ethyl, n-butyl, t-butyl, n-hexyl, benzyl, phenyl, vinyl, allyl, and ethynyl. In other aspects, R^{x} can be selected from methyl, ethyl, n-propyl, n-butyl, sec-butyl, t-butyl, 3-butenyl, n-hexyl, and substituted or unsubstituted phenyl.

Ar can have one R^{x} substituent, or two R^{x} substituents, or three R^{x} substituents, or four R^{x} substituents, and so forth, as appropriate according to the size of the Ar group as discussed above. For example, where Ar is phenyl, the metallocene compound may have as many as five R^{x} substituents. As stated above, R^{x} may be substituted at any position of Ar, and so in some non-limiting aspects, the Ar—R^{x} can be a 2-R^{x}-phenyl group, a 4-R^{x}-phenyl group, a 2,6-di-R^{x}-phenyl group, or a 2,4,6-tri-R^{x} phenyl group.

Ar also can be further substituted, for instance by any appropriate number of halogen substituents. In certain aspects, Ar can be substituted by one or more R^{x} as described above, and can further comprise one or more substituents (e.g., one halogen substituent, or two halogen substituents, or three halogen substituents, or four halogen substituents). In such aspects, each halogen substituent can be F. Thus, Ar—R^{x} can be further substituted by one F, or two F, or three F, or four F, and any position of Ar. In certain aspects, Ar—R^{x} can be a 2,6-difluoro-4-R^{x}-aryl group, a 2,3,5,6-tetrafluoro-4-R^{x}-aryl group, a 2-6-di-R^{x}-4-fluoroaryl group, and so forth, including those represented as below:

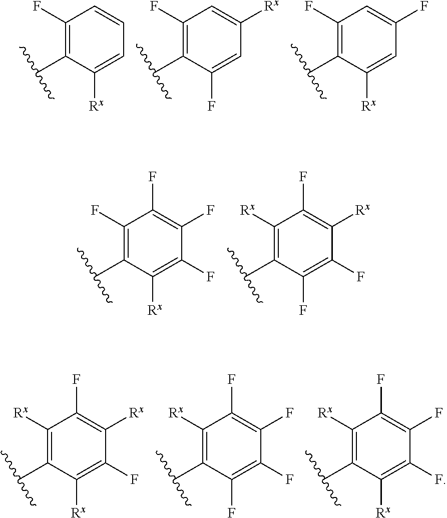

Metallocene compounds also can comprise a hydrocarbon linker between Ar and CP^{A} groups as represented in Formula (I) as —(CH₂)ₙ—. The length of the hydrocarbon linker is not limited to any particular length, and thus in certain aspects, n can be an integer in a range from 0 to 5 (e.g., 0, 1, 2, 3, 4, or 5). In certain aspects, n can be 0. In other aspects, n can be 1.

Illustrative and non-limiting examples of unbridged metallocene compounds having formula (I) and/or which can be prepared according to methods described herein can, in certain aspects, include the following 4-substituted metallocene compounds:

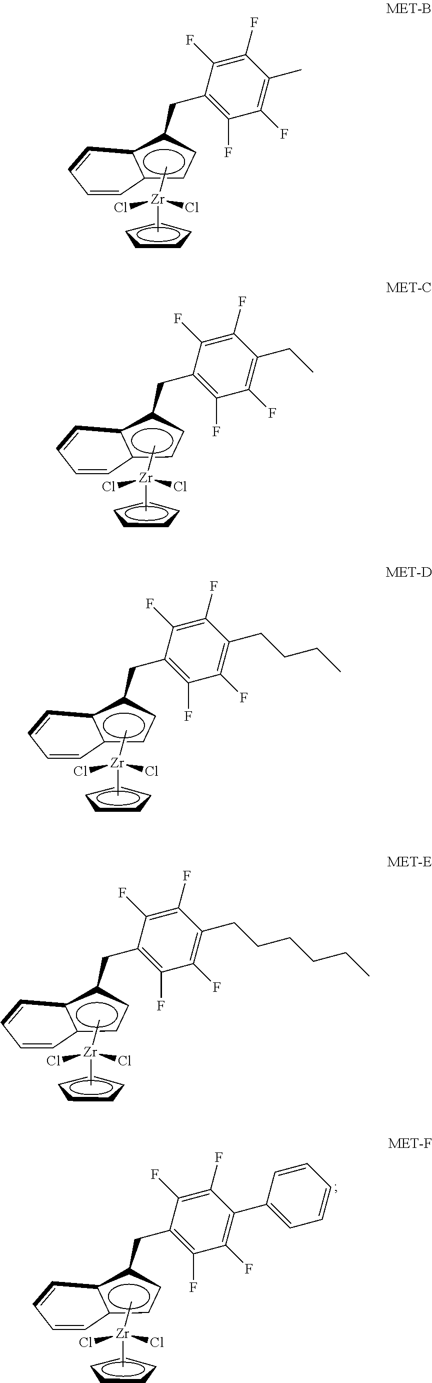

and the like, 2- and 6-monosubstituted analogs of MET-B through MET-F, disubstituted and trisubstituted analogs, and combinations thereof. Additional metallocenes having alternate arrangements of R^{x} and halide substituents, as represented above, also are contemplated herein, as would be understood by those of skill in the art.

As discussed above, substitutions of aryl halogens (e.g., Ar substituents) with a hydrophobic organic substituent (e.g., R^{x}) are surprisingly shown to improve solubility of the metallocene compound without causing significant variation in the catalytic activity or in the properties of oligomer and polymer products formed by respective oligomerization and polymerization processes employing catalyst compositions comprising the metallocene compounds. In certain aspects, metallocene compounds as described herein can have a solubility at 25° C. in 1-decene of at least 0.01 wt. %, at least 0.05 wt. %, or at least 0.1 wt. %, or at least 0.2 wt. %. In other aspects, metallocene compounds disclosed herein can have a solubility at 25° C. in 1-decene in a range from 0.01 wt. % to 2 wt. %, from 0.1 wt. % to 1 wt. %, from 0.1 wt. % to 0.5 wt. %, from 0.2 wt. % to 1 wt. %, or from 0.2 wt. % to 0.5 wt. %.

The solubility of metallocene compounds also can be measured as relative to metallocenes lacking the R^{x} substitution and having an F in its place. In certain aspects, metallocene compounds can have a solubility at 25° C. in 1-decene at least 10% greater than, at least 25% greater than, at least 50% greater than, at least 100% greater than, or at least three times greater than that of an otherwise identical metallocene compound wherein each R^{x} is F. In other aspects, metallocene compounds can have a solubility at 25° C. in 1-decene in a range from 50% to 500% greater, or from 100% to 300% greater than that of an otherwise identical metallocene compound wherein each R^{x} is F.

### Catalyst Compositions

In accordance with aspects of the present invention, metallocene compounds disclosed herein can be employed within catalyst compositions, e.g., for oligomerization and polymerization processes as described below.

Generally, catalyst compositions disclosed herein can comprise any of the metallocene compounds above, an activator, and optionally, a co-catalyst. In certain aspects, the activator can comprise an aluminoxane compound, an organoboron or organoborate compound, an ionizing ionic compound, a chemically treated solid oxide, or a combination thereof. When the activator in the catalyst composition is a chemically treated solid oxide (activator), then aluminoxane, organoboron or organoborate, and ionizing ionic materials, if present, are referred to as co-catalysts. In certain aspects, one or more than one metallocene compound, activator, or co-catalyst can be present in the catalyst composition. For example, catalyst compositions can further comprise a second metallocene compound (e.g., a bridged metallocene).

Aluminoxanes that can Serves activators (and co-catalysts) in this disclosure are generally represented by formulas such as (R³—Al—O)ₙ, R³(R³—Al—O)ₙAl(R³)₂, and the like, wherein the R³ group is typically a linear or branched C₁-C₆ alkyl such as methyl, ethyl, propyl, butyl, pentyl, or hexyl wherein n typically represents an integer from 1 to 50. In one aspect, the aluminoxane compound used in the disclosed catalyst composition can include, but is not limited to, methylaluminoxane (MAO), ethylaluminoxane, modified methylaluminoxane (MMAO) such as an isobutyl-modified methyl aluminoxane, n-propylaiuminoxane, iso-propylaluminoxaie, n-butylaluminoxane, t-butyl-aluminoxane, sec-butylaluminoxane, iso-butylaluminoxane, t-butyl aluminoxane, 1-pentyl-aluminoxane, 2-pentylaluminoxane, 3-pentylaluminoxane, iso-pentylaluminoxane, neopentylaluminoxane, or combinations thereof.

While aluminoxanes with different types of “R” groups such as R³ are encompassed by the present disclosure, methyl aluminoxane (MAO), ethyl aluminoxane, or isobutyl aluminoxane are typical aluminoxane activators used in the catalyst compositions of this disclosure. These aluminoxanes are prepared from trimethylaluminum, triethylaluminum, or triisobutylaluminum, respectively, and are sometimes referred to as poly(methylaluminum oxide), poly(ethylaluminum oxide), and poly(isobutylaluminum oxide), respectively. It is also within the scope of the disclosure to use an aluminoxane in combination with a trialkylaluminum, such as disclosed in U.S. Pat. No. 4,794,096.

Organoboron compounds that can be used in the catalyst composition of this disclosure are similarly varied. In one aspect, the organoboron compound can comprise neutral boron compounds, borate salts, or combinations thereof. For example, the organoboron compounds of this disclosure can comprise a fluoroorgano boron compound, a fluoroorgano borate compound, or a combination thereof. Any fluoroorgano boron or fluoroorgano borate compound known in the art can be utilized. The term fluoroorgano boron compound has its usual meaning to refer to neutral compounds of the form BY₃. The term fluoroorgano borate compound also has its usual meaning to refer to the monoanionic salts of a fluoroorgano boron compound of the form [cation]+[BY₄]⁻, where Y represents a fluorinated organic group. For convenience, fluoroorgano boron and fluoroorgano borate compounds are typically referred to collectively by organoboron and organoborate compounds, or by either name as the context requires.

Examples of organoboron or organoborate compounds that can be used as activators in the present disclosure include, but are not limited to, fluorinated aryl borates such as, N,N-dimethylanilinium tetrakis(pentafluorophenyl)borate (DTPB), triphenylcarbenium tetrakis(pentafluorophenyl)borate, lithium tetrakis(pentafluorophenyl)borate, N,N-dimethylanilinium tetrakis[3,5-bis(trifluoro-methyl)phenyl]borate, triphenylcarbenium tetrakis[3,5-bis(trifluoromethyl)phenyl]borate, and the like, including mixtures thereof; alternatively, N,N-dimethylanilinium tetrakis-(pentafluorophenyl)borate (DTBP); alternatively, triphenylcarbenium tetrakis(pentafluorophenyl)borate; alternatively, lithium tetrakis(pentafluorophenyl)borate; alternatively, N,N-dimethylanilinium tetrakis[3,5-bis(trifluoromethyl)phenyl]borate; or alternatively, triphenylcarbenium tetrakis[3,5-bis(trifluoromethyl)phenyl]borate. Other suitable organoboron or organoborate activators include, but are not limited to, tris(pentafluorophenyl)-boron, tris[3,5-bis(trifluoromethyl)phenyl]boron, and the like, including mixtures thereof.

Although not intending to be bound by the following theory, these examples of organoboron and organoborate compounds, and related compounds, are thought to form “weakly-coordinating” anions when combined with organometal compounds, as disclosed in U.S. Pat. No. 5,919,983.

An ionizing ionic compound is an ionic compound which can function to enhance the activity of the catalyst composition. Examples of ionizing ionic compounds that may be suitable as activators in catalyst compositions disclosed herein include, but are not limited to, the following compounds: tri(n-butyl)ammonium tetrakis(p-tolyl)borate, tri(n-butyl) ammonium tetrakis(m-tolyl)borate, tri(n-butyl)ammonium tetrakis(2,4-dimethylphenyl)borate, tri(n-butyl)ammonium tetrakis(3,5-dimethylphenyl)borate, tri(n-butyl)ammonium tetrakis[3,5-bis(trifluoromethyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis(2,4-dimethylphenyl)borate, N,N-dimethylanilinium tetrakis(3,5-dimethylphenyl)borate, N,N-dimethylanilinium tetrakis[3,5-bis(trifluoromethyl)phenyl]borate, N,N-dimethylanilinium tetrakis(pentafluorophenyl)borate, triphenylcarbenium tetrakis(p-tolyl)borate, triphenylcarbenium tetrakis(m-tolyl)borate, triphenylcarbenium tetrakis(2,4-dimethylphenyl)borate, triphenylcarbenium tetrakis(3,5-dimethylphenyl)borate, triphenylcarbenium tetrakis[3,5-bis(trifluoromethyl)phenyl] borate, triphenylcarbenium tetrakis(pentafluorophenyl)borate, tropylium tetrakis(p-tolyl)borate, tropylium tetrakis(m-tolyl)borate, tropylium tetrakis(2,4-dimethylphenyl)borate, tropylium tetrakis(3,5-dimethylphenyl)borate, tropylium tetrakis[3,5-bis(trifluoromethyl)phenyl]borate, tropylium tetrakis(pentafluorophenyl) borate, lithium tetrakis(pentafluorophenyl)borate, lithium tetraphenylborate, lithium tetrakis(p-tolyl)borate, lithium tetrakis(m-tolyl)borate, lithium tetrakis(2,4-dimethylphenyl)borate, lithium tetrakis(3,5-dimethylphenyl)borate, lithium tetrafluoroborate, sodium tetrakis(pentafluorophenyl)borate, sodium tetraphenylborate, sodium tetrakis(p-tolyl)borate, sodium tetrakis(m-tolyl)borate, sodium tetrakis(2,4-dimethylphenyl)borate, sodium tetrakis(3,5-dimethylphenyl)borate, sodium tetrafluoroborate, potassium tetrakis(pentafluorophenyl)borate, potassium tetraphenylborate, potassium tetrakis(p-tolyl)borate, potassium tetrakis(m-tolyl)borate, potassium tetrakis(2,4-dimethylphenyl)borate, potassium tetrakis(3,5-dimethylphenyl)borate, potassium tetrafluoroborate, lithium tetrakis(pentafluorophenyl)aluminate, lithium tetraphenylaluminate, lithium tetrakis(p-tolyl)aluminate, lithium tetrakis(m-tolyl)aluminate, lithium tetrakis(2,4-dimethylphenyl)aluminate, lithium tetrakis(3,5-dimethylphenyl)aluminate, lithium tetrafluoroaluminate, sodium tetrakis(pentafluorophenyl)aluminate, sodium tetraphenylaluminate, sodium tetrakis(p-tolyl)aluminate, sodium tetrakis(m-tolyl)aluminate, sodium tetrakis(2,4-dimethylphenyl)aluminate, sodium tetrakis(3,5-dimethylphenyl)aluminate, sodium tetrafluoro-aluminate, potassium tetrakis(pentafluorophenyl)aluminate, potassium tetraphenylaluminate, potassium tetrakis(p-tolyl)aluminate, potassium tetrakis(m-tolyl)aluminate, potassium tetrakis(2,4-dimethylphenyl)aluminate, potassium tetrakis (3,5-dimethylphenyl)aluminate, potassium tetrafluoroaluminate, and the like, or combinations thereof. Additional examples and description of ionizing ionic compounds are generally disclosed throughout U.S. Pat. No. 11,186,665.

Chemically treated solid oxides are also suitable activators in the disclosed catalyst compositions. In certain aspects, the chemically treated solid oxides described herein generally can refer to those disclosed, for instance, in U.S. Pat. Nos. 8,536,391 and 10,919,996. In certain aspects, the chemically treated solid oxide can comprise a solid oxide comprising oxygen and at least one element selected from Group 2, 3, 4, 5, 6, 7, 8, 9, 10, 11, 12, 13, 14, or 15 of the periodic table, or comprise oxygen and at least one element selected from the lanthanide or actinide elements; alternatively, the solid oxide can comprise oxygen and at least one element selected from Group 4, 5, 6, 12, 13, or 14 of the periodic table, or comprise oxygen and at least one element selected from the lanthanide elements. (See: Hawley's Condensed Chemical Dictionary, 11th Ed., John Wiley & Sons; 1995; Cotton, F. A.; Wilkinson, G.; Murillo; C. A.; and Bochmann; M. Advanced Inorganic Chemistry, 6th Ed., Wiley-Interscience, 1999.) In some aspects, the inorganic oxide can comprise oxygen and at least one element selected from Al, B, Be, Bi, Cd, Co, Cr, Cu, Fe, Ga, La, Mn, Mo, Ni, Sb, Si, Sn, Sr, Th, Ti, V, W, P, Y, Zn or Zr; alternatively, the inorganic oxide can comprise oxygen and at least one element selected from Al, B, Si, Ti, P, Zn or Zr.

In certain aspects, the chemically treated solid oxide can comprise a solid oxide comprising Al₂O₃, B₂O₃, BeO, Bi₂O₃, CdO, CO₃O₄, Cr₂O₃, CuO, Fe₂O₃, Ga₂O₃, La₂O₃, Mn₂O₃, MoO₃, NiO, P₂O₅, Sb₂O₅, SiO₂, SnO₂, SrO, ThO₂, TiO₂, V₂O₅, WO₃, Y₂O₃, ZnO, ZrO₂, mixed oxides thereof, and combinations thereof. In certain aspects, the solid oxide can comprise silica, alumina, silica-alumina, silica-coated alumina, aluminum phosphate, aluminophosphate, heteropolytungstate, titania, zirconia, magnesia, boria, zinc oxide, a mixed oxide thereof, or any combination thereof. In other aspects, solid oxides can comprise silica-coated alumina.

In certain aspects, the chemically treated solid oxide can comprise a solid oxide treated with at least one electron-withdrawing anion, wherein the solid oxide can comprise any oxide that is characterized by a high surface area, and the electron-withdrawing anion can comprise any anion that increases the acidity of the solid oxide as compared to the solid oxide that is not treated with at least one electron-withdrawing anion.

The solid oxide material can be treated with a source of halide ion, sulfate ion, or a combination thereof, and optionally treated with a metal ion. In one aspect, the solid oxide material can be treated with a source of sulfate (termed a sulfating agent), a source of phosphate (termed a phosphating agent), a source of iodide ion (termed an iodiding agent), a source of bromide ion (termed a bromiding agent), a source of chloride ion (termed a chloriding agent), a source of fluoride ion (termed a fluoriding agent), or any combination thereof, and calcined to provide the chemically treated solid oxide.

In certain aspects, the chemically treated solid oxide can comprise a solid oxide treated with an electron-withdrawing anion, wherein the solid oxide is selected from silica, alumina, silica-alumina, aluminum phosphate, heteropolytungstates, titania, zirconia, magnesia, boria, zinc oxide, mixed oxides thereof, or mixtures thereof, and the electron-withdrawing anion is selected from fluoride, chloride, bromide, phosphate, triflate, bisulfate, sulfate, fluorophosphate, fluorosulfate, or any combination thereof. Thus, in certain aspects, the chemically treated solid oxide can comprise fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof. In certain aspects, the chemically treated solid oxide can comprise fluorided alumina, sulfated alumina, fluorided silica-alumina, sulfated silica-alumina, fluorided silica-coated alumina, fluorided-chlorided silica-coated alumina, sulfated silica-coated alumina, or any combination thereof. In other aspects, the chemically treated solid oxide can comprise sulfated alumina and/or fluorided silica-coated alumina.

In certain aspects, chemically treated solid oxides disclosed herein can comprise a calcined solid oxide. Thus, in this aspect, the solid oxide can be calcined or uncalcined; alternatively, calcined; or alternatively, uncalcined. In certain aspects, the solid oxide can be calcined prior to, during, or after the solid oxide compound is contacted with the electron-withdrawing anion source resulting in the chemically treated solid oxide. Calcining of the treated solid oxide is generally conducted in an ambient atmosphere; alternatively, in a dry ambient atmosphere. The solid oxide can be calcined at a temperature from 200° C. to 900° C.; alternatively, from 300° C. to 800° C.; alternatively, from 400° C. to 700° C.; or alternatively, from 350° C. to 550° C. The period of time at which the solid oxide is maintained at the calcining temperature can be 1 minute to 100 hours; alternatively, from 1 hour to 50 hours; alternatively, from 3 hours to 20 hours; or alternatively, from 1 to 10 hours.

In certain aspects disclosed herein, catalyst composition can further comprise a co-catalyst. In certain aspects, the co-catalyst can comprise an organoaluminum compound, an aluminoxane compound, an organoboron or organoborate compound, an ionizing ionic compound, or a combination thereof; alternatively, the co-catalyst can comprise an organoaluminum compound. In an aspect, suitable organoaluminum compounds can have the formula, (R^{Z})₃Al, wherein each R^{Z} independently can be an aliphatic group having from 1 to 10 carbon atoms. For example, each R^{Z} independently can be methyl, ethyl, propyl, butyl, hexyl, or isobutyl. In another aspect, examples of organoaluminum compounds suitable for use in accordance with the present invention can include, but are not limited to, trialkylaluminum compounds, dialkylaluminum halide compounds, dialkylaluminum hydride compounds, as well as combinations thereof. Specific non-limiting examples of suitable organoaluminum compounds can include trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), and the like, or combinations thereof.

Generally, the organoaluminum compound (or other co-catalyst) can be used in any suitable amount relative to the metallocene compound. In certain aspects, a molar ratio of the co-catalyst to the metallocene compound in the catalyst composition can be in a range from 0.1:1 to 100,000:1, from 1:1 to 10,000:1, from 10:1 to 1,000:1, or from 50:1 to 500:1. Catalyst compositions disclosed herein also may be characterized according to the weight ratio of the metallocene compound to the activator, which in certain aspects can be in a range from 1:10 to 1:10,000, from 1:10 to 1:1,000, from 1:10 to 500:1, or from 1:10 to 1:100.

In another aspect of the present invention, a catalyst composition can be substantially free of aluminoxanes, organoboron or organoborate compounds, ionizing ionic compounds, and/or other similar materials; alternatively, substantially free of aluminoxanes; alternatively, substantially free or organoboron or organoborate compounds; or alternatively, substantially free of ionizing ionic compounds. In these aspects, the catalyst composition has catalyst activity, discussed herein, in the absence of these additional materials. For example, a catalyst composition of the present invention can consist essentially of a metallocene, an activator, and an organoaluminum compound, wherein no other materials are present in the catalyst composition which would increase/decrease the activity of the catalyst composition by more than about 10% from the catalyst activity of the catalyst composition in the absence of said materials.

Catalyst compositions of the present invention generally have a catalyst activity greater than 250 grams of ethylene polymer (homopolymer and/or copolymer, as the context requires) per gram of activator-support per hour (abbreviated g/(g*h)). In another aspect, the catalyst activity can be greater than 350, greater than 450, or greater than 550 g/(g*h). Yet, in another aspect, the catalyst activity can be greater than 700 g/(g*h), greater than 1000 g/(g*h), or greater than 2000 g/(g*h), and often as high as 5000-10,000 g/(g*h). Illustrative and non-limiting ranges for the catalyst activity include from 500 to 5000, from 750 to 4000, or from 1000 to 3500 g/(g*h), and the like. In certain aspects, the activities noted above can be obtained under slurry polymerization conditions, with a triisobutylaluminum co-catalyst, using isobutane as the diluent, at a polymerization temperature of 80° C. and a reactor pressure of 320 psig. Moreover, in some aspects, the activator-support can comprise sulfated alumina, fluorided silica-alumina, or fluorided silica-coated alumina, although not limited thereto.

### Oligomerization Processes

Processes disclosed herein can comprise contacting any catalyst composition disclosed herein with an alpha olefin monomer and optionally H₂ under oligomerization conditions to produce an oligomer product.

A wide range of alpha olefin monomers can be reacted in the processes provided herein. For example, the alpha olefin can comprise, consist essentially of, or consist of, a C₄ to C₃₀ alpha olefin; alternatively, a C₄ to C₁₈ alpha olefin; alternatively, a C₄ to C₁₄ alpha olefin; alternatively, a C₅ to C₁₈ alpha olefin; alternatively, a C₆ to C₁₆ alpha olefin; or alternatively, a C₈ to C₁₂ alpha olefin. In an aspect, the oligomer product can be produced from an alpha olefin comprising, consisting essentially of, or consisting of, a C₆ alpha olefin, a C₈ alpha olefin, a C₁₀ alpha olefin, a C₁₂ alpha olefin, a C₁₄ alpha olefin, a C₁₆ alpha olefin, or any combination thereof; alternatively, a C₈ alpha olefin, a C₁₀ alpha olefin, a C₁₂ alpha olefin, or any combination thereof; alternatively, a C₆ alpha olefin; alternatively, a C₈ alpha olefin; alternatively, a C₁₀ alpha olefin; alternatively, a C₁₂ alpha olefin; alternatively, a C₁₄ alpha olefin; alternatively, a C₁₆ alpha olefin; or alternatively, a C₁₈ alpha olefin. In a further aspect, the alpha olefin can comprise, consist essentially of, or consist of 1-butene, 1-pentene, 1-hexene, 1-octene, 1-decene, 1-dodecene, 1-tetradecene, 1-hexadecene, or any combination thereof. For instance, the alpha olefin can comprise, consist essentially of, or consist of 1-octene; alternatively, 1-decene; or alternatively, 1-dodecene.

The alpha olefin monomers can be derived from ethylene that is produced from fossil-based feedstocks, bio-based feedstocks, or recycled, circular feedstocks that are fossil-based or bio-based. For example, the alpha olefin can be derived from ethylene that is produced from natural gas feedstocks. Alternatively, the alpha olefin can be derived from ethylene produced from naphtha obtained from crude oil. Alternatively, the alpha olefin can be derived from ethylene produced from ethanol, wherein the ethanol is derived from cellulosic or lignocellulosic feedstocks (i.e., sugar cane, corn, etc.). Alternatively, the alpha olefin can be derived from ethylene produced from recycled plastic materials that have been pyrolyzed to form a circular pyrolysis gas or pyrolysis oil feedstock. When a renewable or circular feedstock is used, the resulting products can be certified as circular or renewable products. Any suitable amount of the alpha olefin feed can be a normal alpha olefin. Generally, the alpha olefin contains at least 50 wt. % normal alpha olefin(s), and more often, contains at least 60 wt. %, at least 70 wt. %, at least 80 wt. %, at least 85 wt. %, at least 90 wt. %, at least 92.5 wt. %, or at least 95 wt. % of normal alpha olefin(s), and in some aspects, less than or equal to 99.9 wt. %, less than or equal to 99.5 wt. %, less than or equal to 97 wt. %, or less than or equal to 95 wt. % of normal alpha olefin(s), and in other aspects, a range from any minimum amount disclosed herein to any maximum amount disclosed herein of normal alpha olefin(s), e.g., the alpha olefin contains from 85 wt. % to 95 wt. % or from 90 wt. % to 99 wt. %, of 1-hexene, 1-octene, 1-decene, 1-dodecene, or 1-tetradecene. Thus, mixtures of various alpha olefins (or normal alpha olefins) having different numbers of carbon atoms can be used, or alpha olefins (or normal alpha olefins) having predominantly a single number of carbon atoms can be used. While a mixture of different carbon number olefins can be utilized, the processes disclosed herein are particularly well suited for use with alpha olefins (or normal alpha olefins) having a single carbon number.

In an aspect, the alpha olefin monomer can be a C₁₀ mono-olefin mixture comprising 2-butyl-1-hexene, 3-propyl-1-heptene, 4-ethyl-1-octene, 5-methyl-1-nonene, or any combination thereof. In an aspect, the alpha olefin monomer can also contain C₁₄ mono-olefins. In a further aspect, an alpha olefin feed suitable for use in the processes described herein is described in U.S. Pat. No. 10,435,336.

Certain ratios of components may be used to control the oligomerization process. For instance, increasing the weight ratio of the metallocene compound in the catalyst composition to the alpha olefin monomer can lead to a higher conversion, but may also lead to a heavier mixture of oligomer products (e.g., less of the desirable dimer and trimer products). Nonetheless, the catalyst composition and alpha olefin monomer can be contacted at a weight ratio of the metallocene compound to the alpha olefin monomer ranging from 1:100 to 1:1,000,000, from 1:1,000 to 1:1,000,000, from 1:1,000 to 1:500,000, or from 1:10,000 to 1:250,000, although not limited thereto.

The activity of the catalyst composition is relatively high. For instance, the activity can be at least 50,000 g oligomer/g metallocene compound per hour (g/(g*h)), or from 20,000 g/(g*h) to 180,000 g/(g*h), from 40,000 g/(g*h) to 160,000 g/(g*h), or from 60,000 to 120,000 g/(g*h), for instance in aspects where the oligomerization conditions comprise an oligomerization temperature of 110° C., and wherein the catalyst composition comprises a TIBA co-catalyst.

As described herein, the catalyst activities of catalyst compositions, unexpectedly, can be comparable to, or greater than that of otherwise identical catalyst compositions comprising a metallocene compound where each R^{x} is F, when tested and compared under the same oligomerization conditions. Thus, the disclosed oligomerization processes (or catalyst compositions) can be characterized by an activity of the catalyst composition that is comparable to (e.g., within 20%, 15%, 10%, or 5% more or less than) that of an otherwise identical catalyst system comprising a metallocene compound where each R^{x} is F, under the same catalyst preparation and oligomerization conditions.

Oligomerization conditions utilized in the oligomerization processes can comprise an oligomerization temperature from −10° C. to 250° C., from 20° C. to 180° C., from 50° C. to 160° C.; alternatively, from 55° C. to 160° C.; alternatively, from 60° C. to 155° C.; alternatively, from 65° C. to 150° C.; alternatively, from 70° C. to 140° C.; or alternatively, from 75° C. to 140° C. In another non-limiting aspect, the oligomerization temperature from can range from 70° C. to 90° C.; alternatively, from 90° C. to 120° C.; or alternatively, from 110° C. to 140° C.

In another non-limiting aspect, the oligomerization conditions utilized in the oligomerization processes disclosed herein can comprise performing the oligomerization reaction in the presence of hydrogen. The hydrogen partial pressure in the oligomerization reaction can be any pressure of hydrogen that does not adversely affect the oligomerization reaction. In some non-limiting aspects, the oligomerization conditions can include a partial pressure of hydrogen at least 0.1 psig and often up to and including a partial pressure of 50 psig. Typical ranges for the hydrogen partial pressure can include from 0.1 psig to 50 psig, from 0.1 psig to 20 psig, from 0.1 psig to 10 psig, from 1 psig to 20 psig, from 1 psig to 10 psig, from 2 psig to 20 psig, or from 2 psig to 10 psig.

The oligomerization processes, in certain aspects, can further comprise a step of separating at least a portion of the catalyst composition from the oligomer product using any suitable technique, e.g., by filtration. Likewise, the oligomerization processes can further comprise a step of separating unreacted alpha olefin monomer from the oligomer product using any suitable technique, e.g., wiped film evaporating, distillation, short path distillation, or any combination thereof. Optionally, the oligomerization processes can further comprise recycling either or both of the recovered catalyst composition and the recovered unreacted alpha olefin monomer, for instance, for re-use in the oligomerization processes.

Further still, the oligomerization processes, in certain aspects, can comprise a step of fractionating the oligomer product into alpha olefin dimer, alpha olefin trimer, and alpha olefin heavies (including alpha olefin tetramer and higher oligomers), using any suitable technique, e.g., wiped film evaporating, distillation, short path distillation, or any combination thereof. Similarly, the oligomerization processes can further comprise a step of hydrogenating at least a portion of the oligomer product (e.g., alpha olefin trimer) to form a polyalphaolefin. The process of fractionating an oligomer product into several oligomer fractions is generally known and techniques and conditions for carrying out the fractionation, and subsequent separation, purification, and/or hydrogenation steps to transform the oligomer fractions into a polyalphaolefin will be understood by those of skill in the art.

The oligomer product often contains a dimer of the alpha olefin monomer, a trimer of the alpha olefin monomer, and higher molecular weight oligomers of the alpha olefin monomer (e.g., tetramers and heavies). Advantageously, the disclosed oligomerization processes can produce oligomer products having a relatively high amount of dimers and trimers that can be useful in subsequent reactions and in the production of polyalphaolefins.

The oligomer product formed by the oligomerization processes, therefore, can be characterized by the relative amount of specific oligomers. For instance, it can be beneficial to maximize the amount of dimer and trimer, while minimizing heavier oligomers in the oligomer product. Surprisingly, the oligomerization processes are able to operate at high conversions of the alpha olefin monomer without causing a shift in the resulting oligomer product toward heavier oligomers. In certain aspects, the oligomer product can comprise less than or equal to 20 mol %, less than or equal to 15 mol %, less than or equal to 10 mol %, or less than or equal to 5 mol % tetramer. Additionally, or alternatively, the oligomer product can comprise at least 60 mol %, at least 70 mol %, at least 80 mol %, at least 85 mol %, at least 90 mol %, or at least 95 mol % of dimer and trimer (total). Unreacted alpha olefin monomer is excluded from the compositional breakdown of the oligomer product.

In certain aspects, and beneficially, the dimer may be the majority component of the oligomer product, and the oligomer product can contain at least 30 mol %, at least 40 mol %, at least 50 mol %, at least 55 mol %, at least 60 mol %, at least 65 mol %, at least 70 mol %, or at least 75 mol % alpha olefin dimer, based on total oligomers in the oligomer product, and excluding unreacted alpha olefin monomer.

Often, vinylidene is desirable for its high reactivity relative to internal and branched dimers of the alpha olefin monomer. In certain aspects, the dimer in the oligomer product comprises at least 50 mol %, at least 60 mol %, at least 70 mol %, at least 75 mol %, at least 80 μmol %, at least 85 mol %, at least 90 mol %, or at least 95 mol % vinylidene. It follows then, that the amount of internal olefin within the dimer portion of the oligomer product generally can be less than or equal to 15 mol %, less than or equal to 12 mol %, or less than or equal to 10 mol %.

As for catalyst composition activity described above, the product characteristics of oligomerization processes disclosed herein can be comparable to oligomerization processes which rely on analogous metallocene compounds without the R^{x}-substituent. Thus, in certain aspects, the oligomer product can have a dimer content and character (e.g., an amount of internal, trisubstituted dimer, and/or vinylidene dimer in the dimer product) that is comparable to (e.g., within 20%, 15%, 10%, or 5% more or less than) that of an otherwise identical catalyst system comprising a metallocene compound where each R^{x} is F, under the same catalyst preparation and oligomerization conditions.

The polyalphaolefin may have certain desirable properties. For example, one desirable property which can be achieved by utilizing a separation step or steps is 100° C. kinematic viscosity. A second desirable property which can be achieved by utilizing a separation step or steps is to achieve a desired flash point, A third desirable property which can be achieved by utilizing a separation step or steps is to achieve a desired fire point. A fourth desirable property which can be achieved by utilizing a separation step or steps is to achieve a desired Noack volatility, A fifth desirable property which can be achieved by utilizing a separation step or steps is to achieve a desired pour point. In an embodiment, the separation step(s) can be utilized to remove lower and/or higher molecular weight oligomers to produce an alpha olefin oligormer product, or an alpha olefin oligomer product which will produce a polyalphaolefin, having a desired 100° C. kinematic viscosity, flash point, fire point, Noack volatility, and/or pour point.

### Polymerization Processes

Olefin polymers (e.g., ethylene polymers) can be produced from catalyst compositions disclosed herein using any suitable olefin polymerization process using various types of polymerization reactors, polymerization reactor systems, and polymerization reaction conditions. One such olefin polymerization process for polymerizing olefins in the presence of a catalyst composition of the present invention can comprise contacting the catalyst composition with an olefin monomer and optionally an olefin comonomer (one or more) in a polymerization reactor system under polymerization conditions to produce an olefin polymer (e.g., an ethylene polymer). This invention also encompasses any olefin polymers (e.g., ethylene polymers) produced by any of the polymerization processes disclosed herein.

Olefin monomers that can be employed with catalyst compositions and polymerization processes of this invention typically can include olefin compounds having from 2 to 30 carbon atoms per molecule and having at least one olefinic double bond, such as ethylene or propylene. In an aspect, the olefin monomer can comprise a C₂-C₂₀ olefin; alternatively, a C₂-C₂₀ alpha-olefin; alternatively, a C₂-C₁₀ olefin; alternatively, a C₂-C₁₀ alpha-olefin; alternatively, the olefin monomer can comprise ethylene; or alternatively, the olefin monomer can comprise propylene (e.g., to produce a polypropylene homopolymer or a propylene-based copolymer).

When a copolymer (or alternatively, a terpolymer) is desired, the olefin monomer and the olefin comonomer independently can comprise, for example, a C₂-C₂₀ alpha-olefin. In some aspects, the olefin monomer can comprise ethylene or propylene, which is copolymerized with at least one comonomer (e.g., a C₂-C₂₀ alpha-olefin or a C₃-C₂₀ alpha-olefin). According to one aspect of this invention, the olefin monomer used in the polymerization process can comprise ethylene. In this aspect, the comonomer can comprise a C₃-C₁₀ alpha-olefin; alternatively, the comonomer can comprise 1-butene, 1-pentene, 1-hexene, 1-octene, 1-decene, styrene, or any combination thereof; alternatively, the comonomer can comprise 1-butene, 1-hexene, 1-octene, or any combination thereof; alternatively, the comonomer can comprise 1-butene; alternatively, the comonomer can comprise 1-hexene; or alternatively, the comonomer can comprise 1-octene.

In certain aspects, polymerization processes can comprise contacting the catalyst composition, olefin monomer, and optional comonomer with a diluent. Suitable diluents used in slurry polymerization include, but are not limited to, the monomer being polymerized and hydrocarbons that are liquids under reaction conditions. Examples of suitable diluents include, but are not limited to, hydrocarbons such as propane, cyclohexane, isobutane, n-butane, n-pentane, isopentane, neopentane, and n-hexane, heptanes, octanes, petroleum ether, light naphtha, heavy naphtha, or any combination thereof. Some loop polymerization reactions can occur under bulk conditions where no diluent is used.

As used herein, a “polymerization reactor” includes any polymerization reactor capable of polymerizing (inclusive of oligomerizing) olefin monomers and comonomers (one or more than one comonomer) to produce homopolymers, copolymers, terpolymers, and the like. The various types of polymerization reactors include those that can be referred to as a batch reactor, slurry reactor, gas-phase reactor, solution reactor, and the like, or combinations thereof; or alternatively, the polymerization reactor system can comprise a slurry reactor, a gas-phase reactor, a solution reactor, or a combination thereof. The polymerization conditions for the various reactor types are well known to those of skill in the art. Gas phase reactors can comprise fluidized bed reactors or staged horizontal reactors. Slurry reactors can comprise vertical or horizontal loops. Reactor types can include batch or continuous processes. Continuous processes can use intermittent or continuous product discharge. Polymerization reactor systems and processes also can include partial or full direct recycle of unreacted monomer, unreacted comonomer, and/or diluent.

A polymerization reactor system can comprise a single reactor or multiple reactors (2 reactors, more than 2 reactors) of the same or different type. For instance, the polymerization reactor system can comprise a slurry reactor, a gas-phase reactor, a solution reactor, or a combination of two or more of these reactors. Production of polymers in multiple reactors can include several stages in at least two separate polymerization reactors interconnected by a transfer device making it possible to transfer the polymers resulting from the first polymerization reactor into the second reactor. The desired polymerization conditions in one of the reactors can be different from the operating conditions of the other reactor(s). Alternatively, polymerization in multiple reactors can include the manual transfer of polymer from one reactor to subsequent reactors for continued polymerization. Multiple reactor systems can include any combination including, but not limited to, multiple loop reactors, multiple gas phase reactors, or a combination of loop and gas phase reactors. The multiple reactors can be operated in series, in parallel, or both. Accordingly, the present invention encompasses polymerization reactor systems comprising a single reactor, comprising two reactors, and comprising more than two reactors. The polymerization reactor system can comprise a slurry reactor, a gas-phase reactor, a solution reactor, in certain aspects of this invention, as well as multi-reactor combinations thereof.

According to one aspect, the polymerization reactor system can comprise at least one loop slurry reactor comprising vertical or horizontal loops. Monomer, diluent, catalyst, and comonomer can be continuously fed to a loop reactor where polymerization occurs. Generally, continuous processes can comprise the continuous introduction of monomer/comonomer, a catalyst, and a diluent into a polymerization reactor and the continuous removal from this reactor of a suspension comprising polymer particles and the diluent. Reactor effluent can be flashed to remove the solid polymer from the liquids that comprise the diluent, monomer and/or comonomer. Various technologies can be used for this separation step including, but not limited to, flashing that can include any combination of heat addition and pressure reduction, separation by cyclonic action in either a cyclone or hydrocyclone, or separation by centrifugation.

A typical slurry polymerization process (also known as the particle form process) is disclosed, for example, in U.S. Pat. Nos. 3,248,179, 4,501,885, 5,565,175, 5,575,979, 6,239,235, 6,262,191, 6,833,415, and 8,822,608, each of which is incorporated herein by reference in its entirety.

According to yet another aspect, the polymerization reactor system can comprise at least one gas phase reactor (e.g., a fluidized bed reactor). Such reactor systems can employ a continuous recycle stream containing one or more monomers continuously cycled through a fluidized bed in the presence of the catalyst under polymerization conditions. A recycle stream can be withdrawn from the fluidized bed and recycled back into the reactor. Simultaneously, polymer product can be withdrawn from the reactor and new or fresh monomer can be added to replace the polymerized monomer. Such gas phase reactors can comprise a process for multi-step gas-phase polymerization of olefins, in which olefins are polymerized in the gaseous phase in at least two independent gas-phase polymerization zones while feeding a catalyst-containing polymer formed in a first polymerization zone to a second polymerization zone. Representative gas phase reactors are disclosed in U.S. Pat. Nos. 5,352,749, 4,588,790, 5,436,304, 7,531,606, and 7,598,327, each of which is incorporated by reference in its entirety herein.

According to yet another aspect, the polymerization reactor system can comprise a solution polymerization reactor wherein the monomer/comonomer are contacted with the catalyst composition by suitable stirring or other means. A carrier comprising an inert organic diluent or excess monomer can be employed. If desired, the monomer/comonomer can be brought in the vapor phase into contact with the catalytic reaction product, in the presence or absence of liquid material. The polymerization zone can be maintained at temperatures and pressures that will result in the formation of a solution of the polymer in a reaction medium. Agitation can be employed to obtain better temperature control and to maintain uniform polymerization mixtures throughout the polymerization zone. Adequate means are utilized for dissipating the exothermic heat of polymerization.

The polymerization reactor system can further comprise any combination of at least one raw material feed system, at least one feed system for catalyst or catalyst components, and/or at least one polymer recovery system. Suitable reactor systems can further comprise systems for feedstock purification, catalyst storage and preparation, extrusion, reactor cooling, polymer recovery, fractionation, recycle, storage, loadout, laboratory analysis, and process control. Depending upon the desired properties of the olefin polymer, hydrogen can be added to the polymerization reactor as needed (e.g., continuously or pulsed).

Polymerization conditions that can be controlled for efficiency and to provide desired polymer properties can include temperature, pressure, and the concentrations of various reactants. Polymerization temperature can affect catalyst productivity, polymer molecular weight, and molecular weight distribution. Various polymerization conditions can be held substantially constant, for example, for the production of a particular grade of the olefin polymer (or ethylene polymer). A suitable polymerization temperature can be any temperature below the de-polymerization temperature according to the Gibbs Free energy equation. Typically, this includes from 60° C. to 280° C., for example, or from 60° C. to 120° C., depending upon the type of polymerization reactor(s). In some reactor systems, the polymerization temperature generally can be within a range from 70° C. to 105° C., or from 75° C. to 100° C.

Suitable pressures will also vary according to the reactor and polymerization type. The pressure for liquid phase polymerizations in a loop reactor is typically less than 1000 psig (6.9 MPa). Pressure for gas phase polymerization is usually at 200 to 500 psig (1.4 MPa to 3.4 MPa). In certain aspects, polymerization conditions can comprise a reaction pressure in a range from 200 to 1000 psig. Polymerization reactors can also be operated in a supercritical region occurring at generally higher temperatures and pressures. Operation above the critical point of a pressure/temperature diagram (supercritical phase) can offer advantages to the polymerization reaction process.

Olefin polymers encompassed herein can include any polymer produced from any olefin monomer (and optional comonomer(s)) described herein. For example, the olefin polymer can comprise an ethylene homopolymer, a propylene homopolymer, an ethylene copolymer (e.g., ethylene/α-olefin, ethylene/1-butene, ethylene/1-hexene, or ethylene/1-octene), a propylene copolymer, an ethylene terpolymer, a propylene terpolymer, and the like, including combinations thereof. In one aspect, the olefin polymer can be (or can comprise) an ethylene homopolymer, an ethylene/1-butene copolymer, an ethylene/1-hexene copolymer, or an ethylene/1-octene copolymer, or a combination thereof; or alternatively, an ethylene/1-hexene copolymer. In another aspect, the olefin polymer can be (or can comprise) a polypropylene homopolymer and/or a propylene-based copolymer. In some aspects, the olefin polymer can have a bimodal molecular weight distribution, while in other aspects, the olefin polymer can have a multimodal molecular weight distribution. Yet, in still other aspects, the olefin polymer can have a unimodal molecular weight distribution.

Polymerization processes disclosed herein can produce ethylene polymers having characteristics generally similar to those produced by analogous unsubstituted fluorinated metallocene compounds.

The densities of the ethylene polymers can be greater than or equal to 0.94 g/cm³, for example, greater than or equal to 0.942 g/cm³, or greater than or equal to 0.945 g/cm³. Yet, in particular aspects, the density can be in a range from 0.92 to 0.96, from 0.93 to 0.95, from 0.925 to 0.94, or from 0.93 to 0.94 g/cm³. In an aspect, ethylene polymers can have a number-average molecular weight (Mn) in a range from 5,000 g/mol to 250,000 g/mol, from 10,000 g/mol to 200,000 g, or from 20,000 g/mol to 150,000 g/mol. In other aspects, the ethylene polymer can have a Mw in a range from 50,000 to 700,000, from 75,000 to 500,000, or from 100,000 to 400,000 g/mol. In other aspects, the ethylene polymer can have a ratio of Mw/Mn in a range from 2 to 15, or from 2 to 10. In other aspects, the ethylene polymer can have a melt index in a range from 0 to 20 g/10 min, from 0.01 to 10 g/10 min, or from 0.1 to 5 g/10 min. Alternatively, or additionally, the ethylene polymer can have a HLMI in a range from 0 to 100 g/10 min, less than or equal to 25 g/10 min, less than or equal to 20 g/10 min, or less than or equal to 15 g/10 min.

In certain aspects, ethylene polymers of the present invention can have (or can be characterized by) a density in a range from 0.92 to 0.96 g/cm³, a Mw in a range from 50,000 to 700,000, a Mn in a range from 5,000 to 250,000 g/mol, a ratio of Mw/Mn in a range from 1 to 40, a high load melt index (HLMI) in a range from 0 to 100 g/10 min. In certain aspects, the HLMI of the ethylene polymer can be less than or equal to 25 g/10 min, less than or equal to 20 g/10 min, or less than or equal to 15 g/10 min.

As described herein, the catalyst activities of these catalyst compositions, unexpectedly, can be comparable to, or greater than that of otherwise identical catalyst compositions comprising a metallocene compound where each R^{x} is F, when tested and compared under the same polymerization conditions. Thus, the disclosed polymerization processes (or catalyst compositions) can be characterized by an activity of the catalyst composition that is comparable to (e.g., within 20%, 15%, 10%, or 5% more or less than) that of an otherwise identical catalyst system comprising a metallocene compound where each R^{x} is F, under the same catalyst preparation and polymerization conditions.

### Articles and Products

Articles of manufacture can be formed from, and/or can comprise, the olefin polymers and (e.g., ethylene polymers, ethylene/1-hexene polymers) and olefin oligomers (e.g., 1-decene oligomers) of this invention and products thereof (e.g., polyalphaolefins derived from 1-decene oligomers), and, accordingly, are encompassed herein.

For example, articles which can comprise the polymers of this invention can include, but are not limited to, an agricultural film, an automobile part, a bottle, a container for chemicals, a drum, a fiber or fabric, a food packaging film or container, a food service article, a fuel tank, a geomembrane, a household container, a liner, a molded product, a medical device or material, an outdoor storage product (e.g., panels for walls of an outdoor shed), outdoor play equipment (e.g., kayaks, bases for basketball goals), a pipe, a sheet or tape, a toy, or a traffic barrier, and the like. Various processes can be employed to form these articles. Non-limiting examples of these processes include injection molding, blow molding, rotational molding, film extrusion, sheet extrusion, profile extrusion, thermoforming, and the like. Additionally, additives and modifiers often are added to these polymers in order to provide beneficial polymer processing or end-use product attributes. Such processes and materials are described in Modern Plastics Encyclopedia, Mid-November 1995 Issue, Vol. 72, No. 12; and Film Extrusion Manual—Process, Materials, Properties, TAPPI Press, 1992.

In some aspects of this invention, an article of manufacture can comprise any of olefin polymers (or ethylene polymers) described herein, and the article of manufacture can be or can comprise a film, such as a blown film; alternatively, a pipe product; or alternatively, a blow molded product, such as a blow molded bottle.

### EXAMPLES

The invention is further illustrated by the following examples, which are not to be construed in any way as imposing limitations to the scope of this invention. Various other aspects, embodiments, modifications, and equivalents thereof which, after reading the description herein, may suggest themselves to one of ordinary skill in the art without departing from the spirit of the present invention or the scope of the appended claims.

Disclosed are methods for preparing metallocene compounds, and specifically, methods for preparing metallocene compounds by in situ substitution of metallocene precursors as part of a one-pot reaction. Advantageously, the metallocenes prepared by the disclosed methods maintain their catalytic characteristic and function, while improving the solubility in 1-decene relative to otherwise identical unsubstituted metallocene compounds.

### Preparation of Metallocene Compounds

### Prior Art Example 1—Synthesis of Fluorinated Metallocenes (MET-A)

A fluorinated metallocene (denoted MET-A) was prepared for comparison to substituted metallocenes, according to the Reaction Equation 1 below and the experimental procedure that follows. Generally, a substituted indenyl precursor was deprotonated in a first deprotonation step using a single equivalent of n-butyllithium as an organic base. The deprotonated indenyl precursor was then contacted with the metal cyclopentadienyl trichloride complex, prepared as disclosed previously in U.S. Pat. No. 11,186,655 (“IE2”) and generally as shown in the reaction below.

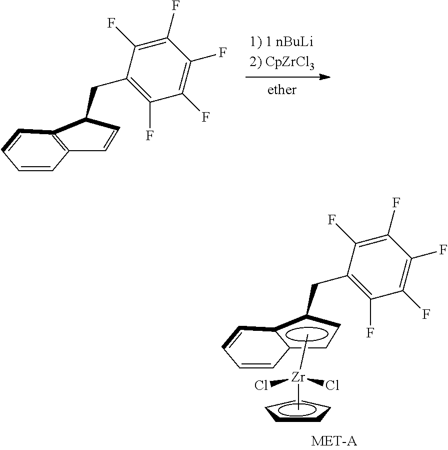

Example 1 (MET-A): A 250 mL flask was charged with 1-((perfluorophenyl)methyl)-1H-indene (1.1 g, 3.8 mol) and 100 mL of diethyl ether. The solution was cooled to −78° C. and nBuLi (1.6 M hexane, 2.4 mL, 3.8 mmol) was added slowly. The resulting solution was allowed to warm to ambient temperature and was stirred 30 minutes. The solution was again cooled to −78° C. and a slurry of cyclopentadienylzirconium(IV) trichloride (1.0 g, 3.8 mmol) in diethyl ether (30 mL) was added. The resulting slurry was allowed to warm to ambient temperature with stirring overnight. The mixture was stripped under high vacuum to a bright yellow solid. The solid was taken up in 30 mL of toluene and centrifuged. The supernatant was transferred to a clean flask and concentrated to about 5 mL. The solution was layered with about 10 mL pentane and placed in a freezer at −35° C. MET-A was isolated as yellow needles with a yield of 0.659 g. ¹H NMR (300 MHz, C₆D₆) δ=7.74 (d, 1H, indene), 7.02 (m, 1H, indene), 6.92 (m, 1H, indene), 6.77 (m, 1H, indene), 6.59 (d, 1H, indene), 5.71 (s, 5H, cyclopentadiene), 4.15 (dd, 2H, —CH₂C₆F₅). ¹⁹F NMR (282.4 MHz, C₆D₆, 25° C.) δ=−144.75 (m), −158.70 (t), −164.01 (m).

### Examples 2-6—One Pot Alkylation of Metallocene Precursors

Surprisingly, it was found that a hydrophobic substituent can be incorporated within the Cp^{A} metallocene precursor by using an additional equivalent of the Bronsted base as a substitution reagent, as shown in the reaction equation below. The reaction can proceed under identical conditions as the deprotonation using the organic base, and therefore allows incorporation of the hydrophobic R^{x} group into the fluorinated Cp^{A} precursor, in situ, as part of a one-pot synthesis of the metallocene compound.

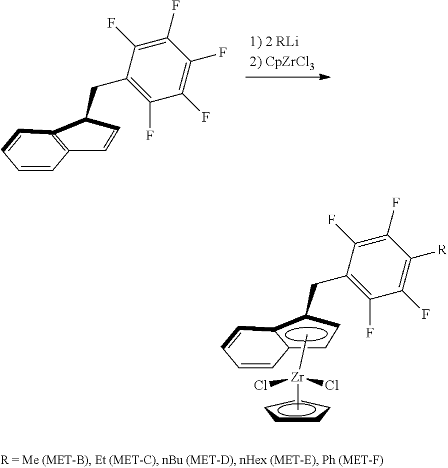

Example 2 (MET-B): A 200 mL flask was charged with 1-((perfluorophenyl)methyl)-1H-indene (850 mg, 2.87 mmol) and 100 mL diethyl ether. The mixture was cooled to −20° C. and MeLi (4.1 mL, 6.6 mmol) was added. The mixture was warmed to ambient temperature with stirring overnight. After 14 hours, a separate flask was charged with CpZrCl₃ (75 mg, 2.87 mmol) and 50 mL diethyl ether. The resulting slurry was cooled to −78° C. and the first reaction mixture was added rapidly. The mixture was slowly warmed to ambient temperature with stirring overnight. After 16 hours, the diethyl ether was removed from the mixture by evaporation at room temperature to yield a yellow solid. The solid was taken up in 50 μmL toluene, centrifuged, and the supernatant was decanted. The resulting solution was concentrated to 20 mL, layered with 10 mL hexane, and placed in a freezer at −30° C. to yield the product as a yellow solid. Yield=0.156 g. ¹H NMR (300 MHz, C₆D₆, 25° C.) δ=7.85 (d, 1H, Indene), 6.98 (d, 1H, Indene), 6.91 (t, 1H, Indene), 6.78 (t, 1H, Indene), 6.67 (m, 1H, Indene), 5.75 (s, 5H, Cp), 5.74 (m, 1H, Indene), 4.51 (d, 1H, Benzyl), 4.22 (d, 1H, Benzyl), 1.65 (t, 3H, Me). ¹⁹F NMR (282.4 MHz, C₆D₆, 25° C.) δ=−145.76 (m), −146.34 (m).

Example 3 (MET-C): A 200 mL flask was charged with 1-((perfluorophenyl)methyl)-1H-indene (1.0 g, 3.38 mmol). The solids were dissolved in 50 mL toluene and 10 mL diethyl ether. The solution was cooled to −20° C. and ethyllithium (15.5 mL, 0.5 M solution, 7.76 mmol) was added slowly. The mixture was allowed to warm to ambient temperature and stirred overnight. A separate flask was charged with CpZrCl₃ (0.88 g, 3.4 mmol) and 20 mL toluene. The mixture was cooled to −78° C. and the first reaction mixture was added. The resulting slurry was allowed to warm to ambient temperature with stirring overnight. The mixture was then concentrated to 40 mL under high vacuum and centrifuged. The orange supernatant was isolated and stripped of solvent by rotoevaporation at room temperature to yield an oily paste. The paste was the taken up in 15 mL toluene and filtered to yield an orange filtrate. The solution was layered with 6 mL pentane and placed in a freezer at −20° C. to crystallize. Several crops of yellow solid yielded 0.319 g. ¹H NMR (300 MHz, CD₂Cl₂, 25° C.) δ=7.78 (d, 1H, Indene), 7.63 (m, 1H, Indene), 7.30 (m, 2H, Indene), 6.77 (m, 1H, Indene), 6.49 (m, 1H, Indene), 6.22 (s, 5H, Cp), 4.45 (d, 1H, Benzyl), 4.20 (d, 1H, Benzyl), 2.69 (m, 2H, Ethyl), 1.17 (t, 3H, Ethyl). ¹⁹F NMR (282.4 MHz, C₆D₆, 25° C.) δ=−145.76 (m), −147.62 (m).

Example 4 (MET-D): A 200 mL flask was charged with 100 mL diethyl ether and 1.13 g (3.81 mmol) of 1-((perfluorophenyl)methyl)-1H-indene. The solution was cooled to −10° C. and nBuLi (5.4 mL, 8.6 mmol, 2.3 eq) was added. The resulting mixture was allowed to warm to ambient temperature. After 2 hours, the dark solution was added to a second flask containing CpZrCl₃ (1.0 g, 3.8 mmol) suspended in 50 mL diethyl ether at −78° C. The resulting slurry was allowed to warm to ambient temperature with stirring. After 16 hours, the mixture was stripped to a paste and 50 mL toluene was added. The yellow slurry was filtered through celite and stripped to a tan oil under high vacuum. The resulting oil was taken up in 40 mL hexane, forming a yellow slurry. The slurry was filtered, and the filtrate was placed in a freezer at −30° C. Several crops of pale-yellow solid were collected. Yield=0.576. ¹H NMR (300 MHz, C₆D₆, 25° C.) δ=7.84 (d, 1H, Indene), 6.96 (d, 1H, Indene), 6.89 (t, 1H, Indene), 6.76 (t, 1H, Indene), 6.66 (m, 1H, Indene), 5.74 (s, 5H, Cp), 5.70 (m, 1H, Indene), 4.52 (d, 1H, Benzyl), 4.25 (d, 1H, Benzyl), 2.34 (t, 2H, Butyl), 1.26 (m, 2H, Butyl), 1.06 (m, 2H, Butyl), 0.70 (t, 3H, Butyl). ¹⁹F NMR (282.4 MHz, C₆D₆, 25° C.) δ=−145.76 (m), −146.78 (m).

Example 5 (MET-E): A 200 mL flask was charged with 1.00 g (3.38 mmol) 1-((perfluorophenyl)methyl)-1H-indene and 100 mL ether. The solution was cooled to −20° C. and n-hexyllithium (3.38 mL, 7.76 mmol) was added. The cold bath was removed, and the reaction mixture was allowed to warm to ambient temperature with stirring. After 1.5 hours, a separate flask was charged with CpZrCl₃ (0.88 g, 3.4 mmol) and 30 mL diethyl ether. The slurry was cooled to −78° C. and the first reaction mixture was added over 5 minutes. The resulting dark brown slurry was allowed to slowly warm to ambient temperature with stirring. After stirring for 13 hours, the pale-yellow slurry was stripped under vacuum to a brown oily solid. Hexanes (80 mL) was added, and the resulting slurry was stirred 1.5 hours. Toluene (20 mL) was added, and the slurry was stirred an additional 2 hours. The mixture was then centrifuged, and the supernatant was decanted. The resulting solution was stripped to solids and the solids were then taken up in 10 mL of toluene. The toluene solution was layered with 5 mL hexane and place in a freezer at −30° C. Several fractions of yellow-orange solid afforded 0.261 g. ¹H NMR (300 MHz, C₆D₆, 25° C.) δ=7.85 (d, 1H, Indene), 6.96 (d, 1H, Indene), 6.88 (t, 1H, Indene), 6.76 (t, 1H, Indene), 6.67 (m, 1H, Indene), 5.74 (s, 5H, Cp), 5.70 (m, 1H, Indene), 4.51 (d, 1H, Benzyl), 4.25 (d, 1H, Benzyl), 2.37 (t, 2H, Hexyl), 1.49-1.03 (br m, 8H, Hexyl), 0.81 (t, 3H, Hexyl). ¹⁹F NMR (282.4 MHz, C₆D₆, 25° C.) δ=−145.70 (m), −146.83 (m).

Example 6 (MET-F): A 200 mL flask was charged with 1-((perfluorophenyl)methyl)-1H-indene (0.924 g, 2.61 mmol) and toluene (100 mL). Diethyl ether (10 mL) was added, and the solution was cooled to −20° C. nBuLi (1.6 mL, 2.6 mmol) was added and the mixture was slowly warmed to ambient temperature. After 1.5 hours, the mixture was added to a flask containing CpZrCl₃ (0.682 g, 2.61 mmol) and 25 mL toluene at −78° C. The resulting slurry was allowed to warm to ambient temperature. After 16 hours, the mixture was concentrated under high vacuum to 50 mL and centrifuged. The orange supernatant was collected and stripped to tacky solids. The solids were redissolved in 15 mL toluene, filtered, layered with 6 mL pentane, and placed in freezer at −30° C. to crystallize. Several crops of yellow solid were isolated. Yield=0.336 g. ¹H NMR (300 MHz, CD₂Cl₂, 25° C.) δ=7.82 (m, 1H, Indene), 7.65 (m, 1H, Indene), 7.46 (m, 2H, Indene), 7.42 (m, 3H, Phenyl), 7.32 (m, 2H, Phenyl), 6.83 (m, 1H, Indene), 6.54 (m, 1H, indene), 6.23 (s, 5H, Cp), 4.55 (d, 1H, Benzyl), 4.29 (d, 1H, Benzyl). ¹⁹F NMR (282.4 MHz, C₆D₆, 25° C.) δ=−144.93 (m), −145.87 (m).

### Example 7—Alkylation of Metallocene Precursors (MET-G)

Another potential synthetic route for incorporating hydrophobic substituents to improve solubility is to prepare novel metal coordinate precursors having substituents on the Cp^{B} ring in order to preserve the catalytic activity of the fluorinated metallocene. However, this route can require isolation of new intermediates for each metallocene derivative, which introduces an additional step and reduced yield. For instance, the reaction below requires a first step of isolating the n-butyl-cyclopentadienyl zirconium trichloride as an intermediate to the metallocene preparation as described above for MET-A.

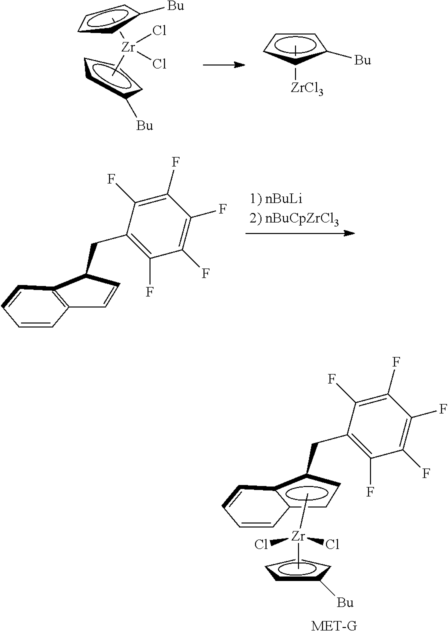

Example 7 (MET-G): A 200 mL flask was charged with 1-((perfluorophenyl)methyl)-1H-indene (1.09 g, 3.68 mmol) and ether (100 mL). nBuLi (2.3 mL, 1.6 M, 3.68 mmol) was added slowly at −20° C. and the mixture was allowed to slowly warm to room temperature. After 2 hours, the mixture was added to a separate flask containing butylcyclopentadienyl zirconium(IV)trichloride (1.17 g, 3.68 mmol) in 30 mL diethyl ether at −78° C. The resulting slurry was allowed to warm to room temperature with vigorous stirring overnight. The solvent was he removed under high vacuum and toluene (40 mL) was added. The slurry was centrifuged and the supernatant was concentrated to approximately 20 mL, layered with hexane, and placed in a freezer at −20° C. Several crops of yellow solid afforded 0.357 g of desired metallocene. ¹H NMR (300 MHz, C₆D₆, 25° C.) δ=7.80 (d, 1H, Indene), 7.03 (d, 1H, Indene), 6.91 (m, 1H, Indene), 6.85 (m, 1H, Indene), 6.70 (m, 1H, Indene), 5.87 (m, 1H, Indene), 5.80 (m, 1H, Cp), 5.61 (m, 1H, Cp), 5.52 (m, 1H, Cp), 5.39 (m, 1H, Cp), 4.39 (d, 1H, Benzyl), 4.12 (d, 1H, Benzyl), 2.52 (m, 2H, Butyl), 1.34 (m, 2H, Butyl), 1.18 (m, 2H, Butyl), 0.80 (t, 3H, Butyl).

### Solubility of Metallocene Compounds

Increased solubility of metallocene compounds in common oligomerization and polymerization solvents (e.g., 1-decene) is advantageous. Surprisingly, metallocene compounds prepared as above demonstrated an improved solubility. The solubility of metallocenes prepared in Examples 1-7 above in 1-decene was examined according to the following procedure: 20 mL vials sealed with crimp caps were charged with 7.40 g (10.0 mL) 1-decene, 7.5 mg of the desired metallocenes, and a small magnetic stir bar. Additional portions of the desired metallocenes were then added in increments of 3-4 mg and the mixture was allowed to stir for 30 minutes after each addition at ambient temperature (21° C.). The resulting mixtures were visually inspected for solids or significant haze. Metallocenes were considered soluble if significant haze or solids were not observed.

TABLE I
Solubility of Metallocenes.

| | | Solubility |
|---|---|---|
| Ex. | MET | (wt. %) |
| 1 | A | <0.1 |
| 2 | B | 0.1 |
| 3 | C | 0.4 |
| 4 | D | 0.4 |
| 5 | E | 0.5 |
| 6 | F | 0.1 |
| 7 | G | 0.4 |

Surprisingly, the solubility of Cp^{A}-R^{x}-substituted metallocenes was unexpectedly improved up to over 4-fold relative to their unsubstituted equivalents in certain instances, as shown in Table I (e.g., MET-C, D, and E). Thus, the solubility of metallocene compounds of Examples 2-7 was able to be improved by an additional in situ substitution that did not require isolation and handling of novel, reactive metallocene intermediates.

### Oligomerization Reactions

Metallocene-catalyzed oligomerizations were performed using MET-A and MET-D as follows. Generally, 1-decene was oligomerized to an oligomer product of dimers, trimers, and tetramers in the presence of MET-A or MET-D, as noted in Table II below. For Examples 8-9, a 1-gallon batch reactor was charged with 675 g of 1-decene. A syringe was charged with CTSO (0.750 g), TIBA (1.3 mL of a 1.0 M hexane solution), and metallocene (5 mg). The chemically treated solid oxide (CTSO) was a fluorided silica-coated alumina (60:40 alumina:silica by weight) containing 4 wt. % F and having a d50 average particle size of 35 microns, a BET surface area of 450 m²/g, and a pore volume of 1.1 mL/g. The catalyst mixture was shaken to mix and was then charged to the reactor under a nitrogen purge. The reactor was heated to 110° C. while stirring at 600-900 rpm. Once the reactor temperature reached setpoint, 633 mg of hydrogen was charged to the reactor. After 1 hour, the reactor was cooled to 35° C. A solution of 10% HCl in isopropyl alcohol (10 mL total charge) was added and the reactor contents were removed.

The reaction mixture was then filtered and analyzed by gas chromatography to determine the yield of oligomer product formed in the reaction mixture, and to determine the relative amount of certain oligomers within the oligomer product (e.g., the trimer:tetramer weight ratio).

Gas chromatographic (GC) analyses were performed using a split injection method on a Bruker 430-GC gas chromatograph with a flame ionization detector (FID). Initial oven temperature was 70° C. for 2 minutes and increased 5° C./min to 290° C. and held for 7 minutes. The column was an all-purpose capillary column (Agilent J&W VF-5 ms, 30 m×0.25 mm×0.25 μm). Data analysis was performed using CompassCDS software.

The distribution of olefin end groups was determined using ¹H NMR on a Bruker 300 MHz NMR. Spectra were recorded in CDCl₃ and are reported relative to SiMe4 as determined by reference to the residual ¹H solvent peak. Integration of the following chemical shift ranges were used to determine the relative amounts of olefin end group: Vinylidene: 4.55-4.75 ppm, Trisubstituted: 4.95-5.15 ppm, Internal: 5.20-5.45 ppm.

The results of oligomerizations of Examples 8-9 are presented in Table II.

Surprisingly, as shown in Table II, the activity observed from oligomerization using the more soluble, R^{x}-substituted metallocene (MET-D) was even higher than that observed for the unmodified fluorinated metallocene (MET-A). More unexpectedly, despite improved activity, the distribution of oligomers in the product mixture was essentially unchanged with MET-D, with only a 0.3% difference in the amount of dimer product, and slight increases in heavy fraction including tetramer. Beneficially, the dimer distribution in the oligomer product was acceptable with 71% of the desired vinylidene produced. While MET-A produced more dimer and more vinylidene in total, the increase in activity and solubility observed by MET-D allows the MET-D oligomerization to have unexpected practical advantages.

TABLE II
Comparison of Oligomer Properties.

| | | Metallocene | Dimer (mol %) | | | | | |
|---|---|---|---|---|---|---|---|---|
| | | Activity | | % | % | % | Trimer | Tetramer |
| Ex. | MET | (g/(g*h)) | Total | internal | trisubstituted | vinylidene | (mol %) | (mol %) |
| 8 | A | 75,060 | 62.3 | 6.3 | 20.7 | 73.0 | 25.8 | 11.8 |
| 9 | D | 108,000 | 62.0 | 9.7 | 19.3 | 71.0 | 25.7 | 12.5 |

### Polymerization Reactions

Polymerization experiments employing catalyst compositions comprising MET-A, MET-B, MET-C, MET-D, MET-E, and MET-F were conducted as follows. Unless otherwise indicated, the polymerization experiments used in the following examples were conducted for 30 μmin in a one-gallon (3.8 L) stainless-steel autoclave reactor containing isobutane as diluent. A syringe was charged with 250 mg solid activator-support, 2 mL hexane, 0.5 mL of 1M TIBA (in hexane) and 2.0 mg metallocene (MET-A, MET-B, MET-C, MET-D, MET-E, or MET-F, as noted in Tables III and IV) in that order. The slurry was charged to the reactor under an isobutane purge. The reactor was sealed, charged with 2 L of isobutane, and heated to 80° C. Ethylene was charged to the reactor and fed on demand to maintain the target pressure of 320 psig (2.2 MPa). 1-Hexene was added as a 12 wt. % feed ratio versus ethylene with 1-hexene feed mass totals as shown in Tables III and IV. The reactor was maintained at the target temperature throughout the experiment by an automated heating-cooling system. After venting of the reactor, purging, and cooling, the resulting polymer product was dried under reduced pressure. For Examples 40-41, the polymerization temperature was 90° C., and ethylene pressure was maintained at 390 psig ethylene, with a 1-hexene feed ratio of 20 wt. % vs. ethylene.

Melt index (MI, g/10 min) was determined in accordance with ASTM D1238 at 190° C. with a 2,160 gram weight, and high load melt index (HLMI, g/10 min) was determined in accordance with ASTM D1238 at 190° C. with a 21,600 gram weight. Density was determined in grams per cubic centimeter (g/cm³) on a compression molded sample, cooled at 15° C. per minute, and conditioned for 40 hours at room temperature in accordance with ASTM D1505 and ASTM D4703.

Molecular weights and molecular weight distributions, where measured, were obtained using a PL-GPC 220 (Polymer Labs, an Agilent Company) system equipped with a IR4 detector (Polymer Char, Spain) and three Styragel HMW-6E GPC columns (Waters, MA) running at 145° C. The flow rate of the mobile phase 1,2,4-trichlorobenzene (TCB) containing 0.5 g/L 2,6-di-t-butyl-4-methylphenol (BHT) was set at 1 mL/min, and polymer solution concentrations were in the range of 1.0-1.5 mg/mL, depending on the molecular weight. Sample preparation was conducted at 150° C. for nominally 4 hr with occasional and gentle agitation, before the solutions were transferred to sample vials for injection. An injection volume of about 200 L was used. The integral calibration method was used to deduce molecular weights and molecular weight distributions using a Chevron Phillips Chemical Company's HDPE polyethylene resin, MARLEX® BHB5003, as the broad standard. The integral table of the broad standard was pre-determined in a separate experiment with SEC-MALS. Mn is the number-average molecular weight, Mw is the weight-average molecular weight, Mz is the z-average molecular weight, and MWD is the ratio of Mw/Mn.

The results of Examples 10-39 are summarized in Table III below. Comparing the MI and density of polymers formed in the presence of MET-A through MET-F and various amounts of 1-hexene comonomer, it was surprisingly found that polymers formed using MET-A through MET-F generally had similar physical properties (e.g., melt indices and density), and similar response to 1-hexene. Thus, it is surprisingly shown that R^{x}-substituted metallocenes can be employed within similar polymerization processes to yield generally similar polymer products. As a result, the advantageous catalytic properties of fluorinated metallocenes, for example, can be preserved in R^{x}-substituted metallocenes that demonstrate improved solubility as shown in Table I above.

TABLE III
Comparison of polymer properties using
alkylated/non-alkylated metallocenes.

| | | | Support | | |
|---|---|---|---|---|---|
| | | 1-Hexene | Activity | MI | Density |
| Entry | MET | (g) | (g/(g*h)) | (g/10 min) | (g/mL) |
| 10 | A | 7.5 | 1,920 | 0.1 | 0.943 |
| 11 | | 12 | 1,776 | 0.2 | 0.94 |
| 12 | | 17.5 | 1,872 | 0.3 | 0.938 |
| 13 | | 30 | 2,144 | 0.3 | 0.937 |
| 14 | B | 0 | 1,840 | 0 | 0.943 |
| 15 | | 7 | 960 | 0.3 | 0.941 |
| 16 | | 17 | 968 | 0.2 | 0.937 |
| 17 | | 35 | 1,536 | 0.5 | 0.934 |
| 18 | | 47 | 1,256 | 0.4 | 0.934 |
| 19 | C | 0 | 1,336 | 0.0 | 0.944 |
| 20 | | 9 | 1,296 | 0.2 | 0.94 |
| 21 | | 19.5 | 1,336 | 0.5 | 0.934 |
| 22 | | 30 | 1,416 | 0.8 | 0.937 |
| 23 | | 46 | 1,248 | 0.9 | 0.934 |
| 24 | D | 0 | 664 | 0.0 | 0.947 |
| 25 | | 6.5 | 560 | 0.1 | 0.943 |
| 26 | | 13 | 576 | 0.3 | 0.939 |
| 28 | | 21 | 648 | 0.3 | 0.938 |
| 29 | | 37 | 712 | 0.7 | 0.936 |
| 30 | E | 0 | 1,144 | 0 | 0.944 |
| 31 | | 8 | 1,056 | 0.2 | 0.94 |
| 32 | | 17 | 1,248 | 0.4 | 0.937 |
| 33 | | 30 | 1,232 | 0.8 | 0.934 |
| 34 | | 64 | 1,880 | 0.5 | 0.936 |
| 35 | F | 0 | 822 | 0 | 0.945 |
| 36 | | 6 | 696 | 0 | 0.944 |
| 37 | | 13.5 | 792 | 0.4 | 0.938 |
| 38 | | 26 | 976 | 0.5 | 0.936 |
| 39 | | 34 | 832 | 0.8 | 0.935 |

Further, Table IV shows that a similar principle applies to metallocenes having R^{x}-substituted Cp^{B} groups, as presented in Example 40 (MET-G). As shown above, while preparation of MET-G requires preparation of novel intermediates, it is shown that Cp^{B} substitutions also can yield highly soluble metallocenes. Surprisingly, Cp^{B}-substituted metallocenes also that exhibit excellent catalytic properties, despite having a greater impact on the product resin properties than observed for MET-A through MET-F (e.g., with respect to MI and density. Given that preparation of Cp^{B} substituted metallocenes also requires MET-G also requires isolation of a novel intermediate, Cp^{A}-substituted metallocenes may offer surprisingly beneficial path to metallocenes with improved solubility.

TABLE IV
Polymer Properties.

| | | | Support | | | | | | |
|---|---|---|---|---|---|---|---|---|---|
| | | 1-Hexene | Activity | MI | d | Mn | Mw | Mz | |
| Ex. | MET | (g) | (g/g/h) | (g/10 min) | (g/mL) | (×10⁻³) | (×10⁻³) | (×10⁻³) | Mw/Mn |
| 39 | A | 49 | 2937 | 0.65 | 0.935 | 66.0 | 139 | 232 | 2.12 |
| 40 | G | 51 | 1392 | 1.5 | 0.941 | 49.8 | 112 | 194 | 2.25 |

The invention is described above with reference to numerous aspects and specific examples. Many variations will suggest themselves to those skilled in the art in light of the above detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A method for the preparation of a metallocene compound, the method comprising: (i) contacting a compound having formula Cp^{A}-(CH₂)ₙ—Ar—X with a Bronsted base to form a deprotonated compound; (ii) contacting the deprotonated compound with a substitution reagent to form a substituted compound having formula Cp^{A}-(CH₂)ₙ—Ar—R^{x}; and (iii) contacting the substituted compound with a second compound having formula Cp^{B}-M-X₃ to form a metallocene compound having formula (I):

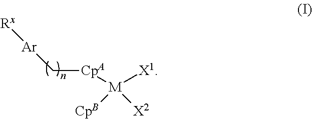

wherein: M is Zr, Ti, or Hf; each X independently is a halogen or NR^{y}₂; X¹ and X² each independently is a monoanionic ligand; Cp^{A} is a cyclopentadienyl, indenyl, or fluorenyl group, optionally substituted with one or more other substituents; Cp^{B} is a substituted or unsubstituted cyclopentadienyl, indenyl, or fluorenyl group; Ar is an aryl group comprising a halogen substituent; R^{x} is a C₁ to C₁₈ hydrocarbyl group substituent on Ar (e.g., selected from alkyl or alkenyl or aryl; a phenyl group, a benzyl group, a C₁ to C₈ alkyl group, or a C₃ to C₈ alkenyl group); n is an integer from 0 to 5; and R^{y} is a C₁ to C₈ hydrocarbyl group.

Aspect 2. The method of aspect 1, wherein the Bronsted base is selected from an organolithium reagent (e.g., methyllithium ethyllithium, n-butyl lithium, t-butyllithium, n-hexyllithium, benzyllithium, phenyllithium) and an organomagnesium halide (e.g., methylmagnesium bromide, methylmagnesium chloride, ethylmagnesium bromide, isopropylmagnesium chloride, t-butyl magnesium chloride, vinylmagnesium bromide, allylmagnesium bromide, ethynylmagnesium chloride, phenylmagnesium chloride, benzylmagnesium chloride).

Aspect 3. The method of aspect 1 or 2, wherein the substitution reagent is selected from an organolithium reagent (e.g., methyllithium ethyllithium, n-butyl lithium, t-butyllithium, n-hexyllithium, benzyllithium, phenyllithium) and an organomagnesium halide (e.g., methylmagnesium bromide, methylmagnesium chloride, ethylmagnesium bromide, isopropylmagnesium chloride, t-butyl magnesium chloride, vinylmagnesium bromide, allylmagnesium bromide, ethynylmagnesium chloride, phenylmagnesium chloride, benzylmagnesium chloride).

Aspect 4. The method of any one of aspects 1-3, wherein the Bronsted base and the substitution reagent each are an organolithium reagent (e.g., methyllithium, n-butyllithium, n-hexyllithium).

Aspect 5. The method of any one of aspects 1-4, wherein the Bronsted base and the substitution reagent are the same.

Aspect 6. The method of any one of aspects 1-5, wherein steps (i) and (ii) are conducted in a one-pot synthesis.

Aspect 7. The method of any one of aspects 1-6, wherein each of steps (i)-(iii) are conducted in a one-pot synthesis.

Aspect 8. A metallocene compound having formula (I):

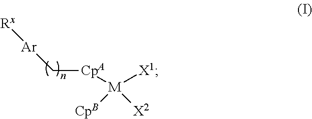

wherein: M is Zr, Ti, or Hf; X¹ and X² independently are a monoanionic ligand (e.g., selected from H, a halide, a C₁ to C₃₆ hydrocarbyl group, or a C₁ to C₃₆ hydrocarboxy group, (or a C₁ to C₃₆ hydrocarbylaminyl group, a C₁ to C₃₆ hydrocarbylsilyl group, a C₁ to C₃₆ hydrocarbylaminylsilyl group, —OBR¹₂, or —OSO₂R¹, wherein R¹ is a C₁ to C₃₆ hydrocarbyl group); Cp^{A} is a cyclopentadienyl, indenyl, or fluorenyl group with substituent —(CH₂)ₙArR^{x}, and optionally substituted with one or more other substituents; Cp^{B} is a substituted or unsubstituted cyclopentadienyl, indenyl, or fluorenyl group; Ar is an aryl group comprising a halogen substituent; R^{x} is a C₁ to C₁₈ hydrocarbyl group substituent on Ar (e.g., selected from alkyl or alkenyl or aryl; a phenyl group, a benzyl group, a C₁ to C₈ alkyl group, or a C₃ to C₈ alkenyl group); and n is an integer from 0 to 5.

Aspect 9. The metallocene compound of aspect 8, wherein X¹ and X² independently are H, F, Cl, Br, a C₁ to C₁₂ hydrocarbyl group, a C₁ to C₁₂ hydrocarboxy group, a C₁ to C₁₂ hydrocarbylaminyl group, a C₁ to C₁₂ hydrocarbylsilyl group, a C₁ to C₁₂ hydrocarbylaminylsilyl group, —OBR₂, or —OSO₂R¹, wherein R¹ is a C₁ to C₁₂ hydrocarbyl group.

Aspect 10. The metallocene compound of aspect 8 or 9, wherein Ar is a phenyl group with two, three, or four halogen substituents.

Aspect 11. The metallocene compound of any one of aspects 8-10, wherein each halogen substituent is F.

Aspect 12. The metallocene compound of any one of aspects 8-11, wherein Ar is selected from:

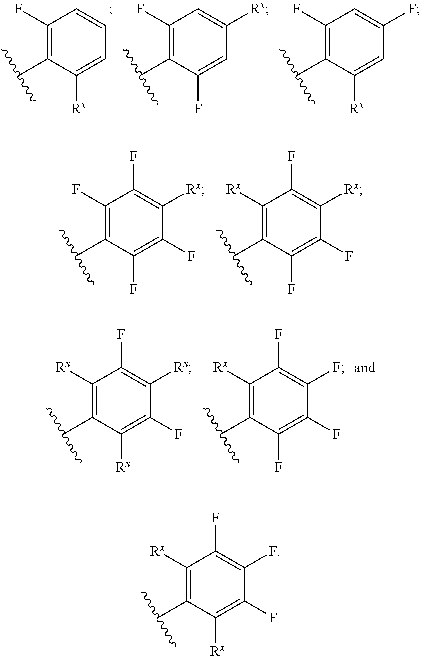

Aspect 13. The metallocene compound of any one of aspects 8-12, wherein R^{x} is selected from methyl, ethyl, n-propyl, n-butyl, sec-butyl, t-butyl, 3-butenyl, n-hexyl, phenyl, and substituted phenyl.

Aspect 14. The metallocene compound of any one of aspects 8-13, wherein n is 0.

Aspect 15. The metallocene compound of any one of aspects 8-13, wherein n is 1.

Aspect 16. The metallocene compound of any one of aspects 8-15, wherein Cp^{A} is substituted with at least one other substituent selected from a C₁-C₁₂ alkyl, C₂-C₁₂ alkenyl, C₆-C₁₀ aryl, or C₇-C₁₂ aralkyl substituent.

Aspect 17. The metallocene compound of any one of aspects 8-16, wherein Cp^{A} is an indenyl group and Cp^{B} is a cyclopentadienyl group.

Aspect 18. The metallocene compound of any one of aspects 8-17, wherein Cp^{B} comprises a C₁-C₁₂ alkyl, C₂-C₁₂ alkenyl, C₆-C₁₀ aryl, or C₇-C₁₂ aralkyl substituent.

Aspect 19. The metallocene compound of any one of aspects 8-18, wherein Cp^{B} comprises substituent —(CH₂)ₙArR^{x}.

Aspect 20. The metallocene compound of any one of aspects 8-17, wherein Cp^{B} is unsubstituted.

Aspect 21. The metallocene compound of any one of aspects 8-20, wherein the metallocene compound is selected from:

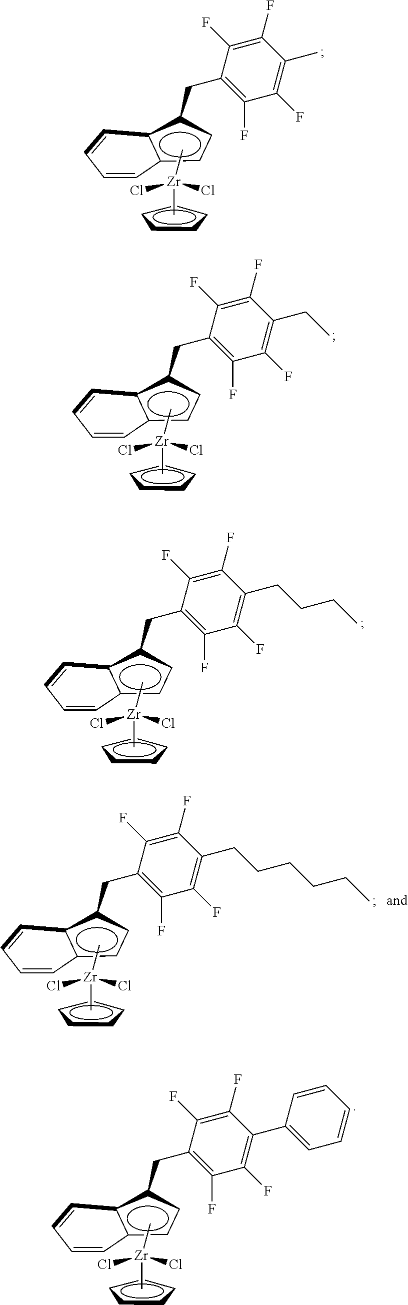

Aspect 22. The metallocene compound of any one of aspects 8-21, having a solubility at 25° C. in 1-decene at least 0.1 wt. % (e.g., from 0.2 wt. % to 2 wt. %, from 0.2 wt. % to 1.0 wt. %, from 0.2 wt. % to 0.5 wt. %).

Aspect 23. The metallocene compound of any one of aspects 8-22, having a solubility at 25° C. in 1-decene greater than (e.g., from 50% to 500% greater, from 100% to 300% greater) that of an otherwise identical metallocene compound wherein each R^{x} is F.

Aspect 24. A catalyst composition comprising: the metallocene compound of any one of aspects 8-23; an activator comprising an aluminoxane compound, an organoboron or organoborate compound, an ionizing ionic compound, a chemically treated solid oxide, or any combination thereof; and optionally, a co-catalyst.

Aspect 25. The catalyst composition of aspect 24, wherein the activator comprises the aluminoxane compound (e.g., methylaluminoxane (MAO), ethylaluminoxane, modified methylaluminoxane (MMAO) such as an isobutyl-modified methyl aluminoxane, n-propylaluminoxane, iso-propylaluminoxane, n-butylaluminoxane, t-butyl-aluminoxane, sec-butylaluminoxane, iso-butylaluminoxane, t-butyl aluminoxane, 1-pentyl-aluminoxane, 2-pentylaluminoxane, 3-pentylaluminoxane, iso-pentylaluminoxane, neopentylaluminoxane, and combinations thereof).

Aspect 26. The catalyst composition of aspect 24 or 25, wherein the activator comprises the organoboron or organoborate compound (e.g., N,N-dimethylanilinium tetrakis(pentafluorophenyl)borate, triphenylcarbenium tetrakis(pentafluorophenyl)borate, lithium tetrakis(pentafluorophenyl)borate, N,N-dimethylanilinium tetrakis[3,5-bis(trifluoro-methyl)phenyl]borate, triphenylcarbenium tetrakis[3,5-bis(trifluoromethyl)phenyl]borate, and combinations thereof).

Aspect 27. The catalyst composition of any one of aspects 24-26, wherein the activator comprises the ionizing ionic compound (e.g., tri(n-butyl)ammonium tetrakis(3,5-dimethylphenyl)borate, tri(n-butyl)-ammonium tetrakis[3,5-bis(trifluoromethyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis[3,5-bis(trifluoro-methyl)phenyl]borate, N,N-dimethylanilinium tetrakis(pentafluorophenyl)borate, triphenyl-carbenium tetrakis(p-tolyl)borate, triphenylcarbenium tetrakis(m-tolyl)borate, triphenylcarbenium tetrakis(2,4-dimethylphenyl)borate, triphenylcarbenium tetrakis(3,5-dimethylphenyl)borate, or triphenylcarbenium tetrakis[3,5-bis(trifluoromethyl)phenyl]borate, lithium tetrakis(p-tolyl)aluminate, lithium tetrakis(m-tolyl)aluminate, lithium tetrakis(2,4-dimethylphenyl)aluminate, lithium tetrakis(3,5-dimethylphenyl)aluminate), or combinations thereof).

Aspect 28. The catalyst composition of any one of aspects 24-27, wherein the activator comprises the chemically treated solid oxide.

Aspect 29. The catalyst composition of any one of aspects 24-28, wherein the chemically treated solid oxide comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided-chlorided silica-coated alumina, fluorided silica-coated alumina, sulfated silica-coated alumina, phosphated silica-coated alumina, or any combination thereof.

Aspect 30. The catalyst composition of any one of aspects 24-29, wherein the chemically treated solid oxide comprises a fluorided solid oxide and/or a sulfated solid oxide.

Aspect 31. The catalyst composition of any one of aspects 24-30, wherein a weight ratio of the metallocene compound to the activator is in any range disclosed herein, e.g., from 1:10 to 1:10,000, from 1:10 to 1:1,000, from 1:10 to 500:1, or from 1:10 to 1:100.

Aspect 32. The catalyst composition of any one of aspects 24-31, wherein the co-catalyst comprises an organoaluminum compound, an aluminoxane compound, an organoboron or organoborate compound, an ionizing ionic compound, or a combination thereof.

Aspect 33. The catalyst composition of any one of aspects 24-32, wherein the co-catalyst comprises an organoaluminum compound.

Aspect 34. The catalyst composition of aspects 32 or 33, wherein the organoaluminum compound comprises trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof.

Aspect 35. The catalyst composition of any one of aspects 24-34, wherein a molar ratio of the co-catalyst to the metallocene compound in the catalyst composition is in any range disclosed herein, e.g., from 0.1:1 to 100,000:1, from 1:1 to 10,000:1, from 10:1 to 1,000:1, or from 50:1 to 500:1.

Aspect 36. The catalyst composition of any one of aspects 28-35, wherein the catalyst composition is substantially free of aluminoxane compounds, organoboron or organoborate compounds, ionizing ionic compounds, or combinations thereof.

Aspect 37. The catalyst composition of any one of aspects 24-36, wherein the catalyst composition further comprises a second metallocene compound.

Aspect 38. An oligomerization process comprising contacting the catalyst composition of any one of aspects 24-37 with an alpha olefin monomer and optionally H₂ under oligomerization conditions to produce an oligomer product.

Aspect 39. The process of aspect 38, wherein the alpha olefin monomer comprises any C₄ to C₁₄ alpha olefin or C₈ to C₁₂ alpha olefin disclosed herein, e.g., 1-octene and/or 1-decene.

Aspect 40. The process of aspect 38 or 39, wherein the alpha olefin monomer comprises a branched alpha olefin.

Aspect 41. The process of any one of aspects 38-40, wherein the alpha olefin monomer comprises a mixture of alpha olefins (e.g., a mixture of C₈ to C₁₂ alpha olefins, or a mixture of C₁₀ alpha olefins).

Aspect 42. The process of any one of aspects 38-41, wherein a weight ratio of the metallocene compound to the alpha olefin monomer is in any range disclosed herein, e.g., from 1:100 to 1:1,000,000, from 1:1,000 to 1:1,000,000, from 1:1,000 to 1:500,000, or from 1:10,000 to 1:250,000.

Aspect 43. The process of any one of aspects 38-42, wherein the oligomerization conditions comprise an oligomerization temperature in any range disclosed herein, e.g., from −10° C. to 250° C., from 20° C. to 180° C., from 50° C. to 160° C., or from 70° C. to 140° C.

Aspect 44. The process of any one of aspects 38-43, wherein the catalyst composition is contacted with the alpha olefin monomer and H₂ at any suitable hydrogen partial pressure (e.g., from 0.1 to 10 psig of H₂).

Aspect 45. The process of any one of aspects 38-44, wherein an activity of the catalyst composition is in any range disclosed herein, e.g., at least 50,000 g oligomer/g metallocene compound per hour (g/(g*h)), from 20,000 g/(g*h) to 180,000 g/(g*h), from 40,000 g/(g*h) to 160,000 g/(g*h), or from 60,000 to 120,000 g/(g*h) under oligomerization conditions comprising an oligomerization temperature of 90° C., and wherein the co-cocatalyst is TIBA.

Aspect 46. The process of any one of aspects 38-45, wherein the activity of the catalyst composition is comparable (e.g., within 20%, 15%, 10%, or 5% more or less than) that of an otherwise identical process employing a catalyst composition comprising a metallocene compound where each R^{x} is F.

Aspect 47. The process of any one of aspects 38-46, further comprising a step of separating at least a portion of the catalyst composition from the oligomer product using any technique disclosed herein, e.g., filtration.

Aspect 48. The process of any one of aspects 38-47, further comprising recycling the separated catalyst composition.

Aspect 49. The process of any one of aspects 38-48, further comprising a step of separating unreacted alpha olefin monomer from the oligomer product using any technique disclosed herein, e.g., wiped film evaporating, distillation, short path distillation, or any combination thereof.

Aspect 50. The process of any one of aspects 38-49, further comprising recycling unreacted alpha olefin monomer.

Aspect 51. The process of any one of aspects 38-50, further comprising a step of fractionating the oligomer product into alpha olefin dimer, alpha olefin trimer, and alpha olefin heavies including alpha olefin tetramer and higher oligomers, using any technique disclosed herein, e.g., wiped film evaporating, distillation, short path distillation, or any combination thereof.

Aspect 52. The process of any one of aspects 38-51, wherein the oligomer product comprises less than or equal to 20 mol %, less than or equal to 15 mol %, less than or equal to 10 μmol %, or less than or equal to 5 mol % tetramer.

Aspect 53. The process of any one of aspects 38-52, wherein the oligomer product comprises at least 60 mol %, at least 70 mol %, at least 80 mol %, at least 85 mol %, at least 90 μmol %, or at least 95 mol % of dimer and trimer (total).

Aspect 54. The process of any one of aspects 38-53, wherein the oligomer product comprises at least 30 mol %, at least 40 mol %, at least 50 mol %, at least 55 mol %, at least 60 μmol %, at least 65 mol %, at least 70 mol %, or at least 75 mol % alpha olefin dimer.

Aspect 55. The process of any one of aspects 38-54, wherein the oligomer product comprises at least 50 mol %, at least 60 mol %, at least 70 mol %, at least 75 mol %, at least 80 μmol %, at least 85 mol %, at least 90 mol %, or at least 95 mol % vinylidene dimer, relative to the amount of dimer in the oligomer product.

Aspect 56. The process of any one of aspects 38-55, wherein the amount of internal olefin within the dimer portion of the oligomer product is less than or equal to 15 mol %, less than or equal to 12 mol %, or less than or equal to 10 mol %.

Aspect 57. The process of any one of aspects 38-56, wherein the oligomer product has a dimer content and character (e.g., the amount of internal, trisubstituted dimer, and/or vinylidene dimer in the dimer product) that is comparable to (e.g., within 20%, 15%, 10%, or 5% more or less than) that of an otherwise identical process employing a catalyst composition comprising a metallocene compound where each R^{x} is F.

Aspect 58. The process of any one of aspects 38-57, further comprising a step of hydrogenating at least a portion of the oligomer product (e.g., alpha olefin trimer) to form a polyalphaolefin.

Aspect 59. The process of aspect 58, wherein the polyalphaolefin has a kinematic viscosity at 100° C. of less than or equal to 20 cSt, 10 cSt, 5 cSt, 4 cSt, or 3 cSt (e.g., in a range from 1 to 10 cSt).

Aspect 60. A polymerization process, the process comprising contacting the catalyst composition of any one of aspects 24-37 with an ethylene monomer and an optional α-olefin comonomer in a polymerization reactor system under polymerization conditions to produce an ethylene polymer.

Aspect 61. The process of aspect 60, wherein the α-olefin comonomer comprises a C₃-C₂₀ α-olefin, or alternatively, a C₃-C₁₀ α-olefin.

Aspect 62. The process of aspect 60 or 61, wherein the α-olefin comonomer comprises 1-butene, 1-hexene, 1-octene, or a mixture thereof.

Aspect 63. The process of any one of aspects 60-62, wherein the polymerization reactor system comprises a batch reactor, a slurry reactor, a gas-phase reactor, a solution reactor, or a combination thereof.

Aspect 64. The process of any one of aspects 60-63, wherein the polymerization reactor system comprises a slurry reactor, a gas-phase reactor, a solution reactor, or a combination thereof.

Aspect 65. The process of any one of aspects 60-64, wherein the polymerization reactor system comprises a loop slurry reactor.

Aspect 66. The process of any one of aspects 60-65, wherein the polymerization reactor system comprises a single reactor.

Aspect 67. The process of any one of aspects 60-65, wherein the polymerization reactor system comprises 2 reactors.

Aspect 68. The process of any one of aspects 60-65, wherein the polymerization reactor system comprises more than 2 reactors.

Aspect 69. The process of any one of aspects 60-68, wherein the polymerization conditions comprise a polymerization reaction temperature in a range from 60° C. to 120° C. (e.g., 80° C.) and a reaction pressure in a range from 200 to 1000 psig (1.4 to 6.9 MPa).

Aspect 70. The process of any one of aspects 60-69, wherein the polymerization conditions are substantially constant, e.g., for a particular polymer grade.

Aspect 71. The process of any one of aspects 60-70, wherein no hydrogen is added to the polymerization reactor system.

Aspect 72. The process of any one of aspects 60-70, wherein hydrogen is added to the polymerization reactor system.

Aspect 73. The process of any one of aspects 60-72, further comprising contacting the catalyst composition, ethylene monomer, an optional α-olefin comonomer with a diluent, e.g., propane, butanes (for example, n-butane, iso-butane), pentanes (for example, n-pentane, iso-pentane), hexanes, heptanes, octanes, petroleum ether, light naphtha, heavy naphtha, or any combination thereof.

Aspect 74. The process of any one of aspects 60-73, wherein the ethylene polymer comprises an ethylene homopolymer, an ethylene/1-butene copolymer, an ethylene/1-hexene copolymer, and/or an ethylene/1-octene copolymer.

Aspect 75. The process of any one of aspects 60-74, wherein the ethylene polymer comprises an ethylene/1-hexene copolymer.

Aspect 76. The process of any one of aspects 60-75, wherein the catalyst composition is characterized by a total metallocene activity in a range from 30,000 g/(g*h) (grams polyethylene per gram of metallocene per hour) to 800,000 g/(g*h).

Aspect 77. The process of any one of aspects 60-76, wherein the activity of the catalyst composition is comparable (e.g., within 20%, 15%, 10%, or 5% more or less than) that of an otherwise identical process employing a catalyst composition comprising a metallocene compound where each R^{x} is F.

Aspect 78. The process of any one of aspects 60-77, wherein the ethylene polymer has a number-average molecular weight (Mn) in a range of from 5,000 g/mol to 250,000 g/mol, from 10,000 g/mol to 200,000 g, or from 20,000 g/mol to 150,000 g/mol.

Aspect 79. The process of any one of aspects 60-78, wherein the ethylene polymer has a Mw in any range disclosed herein, e.g., from 50,000 to 700,000, from 75,000 to 500,000, or from 100,000 to 400,000 g/mol.

Aspect 80. The process of any one of aspects 60-79, wherein the ethylene polymer has a ratio of Mw/Mn in a range from 2 to 15, or from 2 to 10.

Aspect 81. The process of any one of aspects 60-80, wherein ethylene polymer has a density in any range disclosed herein, e.g., from 0.92 to 0.96, from 0.93 to 0.95, from 0.925 to 0.94, or from 0.93 to 0.94 g/cm³.

Aspect 82. The process of any one of aspects 60-81, wherein the ethylene polymer has a melt index in any range disclosed herein, e.g., from 0 to 20 g/10 min, from 0.01 to 10 g/10 μmin, or from 0.1 to 5 g/10 min.

Aspect 83. The polymer of any one of aspects 60-82, wherein the ethylene polymer has a HLMI in any range disclosed herein, e.g., from 0 to 100 g/10 min, less than or equal to 25 g/10 min, less than or equal to 20 g/10 min, or less than or equal to 15 g/10 min.

We claim:

1. A method for the preparation of a metallocene compound, the method comprising:
(i) contacting a first compound having formula Cp^{A}-(CH₂)ₙ—Ar—X with a Bronsted base to form a deprotonated compound;
(ii) contacting the deprotonated compound with a substitution reagent to form a substituted compound having formula Cp^{A}-(CH₂)ₙ—Ar—R^{x}; and
(iii) contacting the substituted compound with a second compound having formula Cp^{B}-M-X₃ to form a metallocene compound having formula (I): 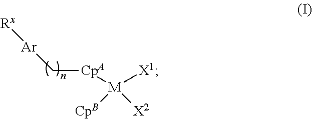
wherein:
M is Zr, Ti, or Hf;
each X independently is a halogen or NR^{y}₂;
X¹ and X² independently are a monoanionic ligand;
Cp^{A} is a cyclopentadienyl, indenyl, or fluorenyl group, optionally substituted with one or more other substituents;
Cp^{B} is a substituted or unsubstituted cyclopentadienyl, indenyl, or fluorenyl group;
Ar is an aryl group comprising a halogen substituent;
R^{x} is a C₁ to C₁₈ hydrocarbyl group substituent on Ar;
n is an integer from 0 to 5; and
R^{y} is a C₁ to C₈ hydrocarbyl group.

2. The method of claim 1, wherein the Bronsted base comprises an organolithium reagent and/or an organomagnesium halide.

3. The method of claim 2, wherein the Bronsted base comprises the organolithium reagent and the organolithium reagent comprises methyllithium, ethyllithium, n-butyl lithium, n-hexyllithium, phenyllithium, or a combination thereof.

4. The method of claim 1, wherein the substitution reagent comprises an organolithium reagent and/or an organomagnesium halide.

5. The method of claim 4, wherein the substitution reagent comprises the organolithium reagent and the organolithium reagent comprises methyllithium, ethyllithium, n-butyl lithium, n-hexyllithium, phenyllithium, or a combination thereof.

6. The method of claim 1, wherein the Bronsted base and the substitution reagent are the same.

7. The method of claim 1, wherein steps (i) and (ii) are conducted in a one-pot synthesis.

8. The method of claim 1, wherein each of steps (i)-(iii) are conducted in a one-pot synthesis.

9. A metallocene compound having formula (I): 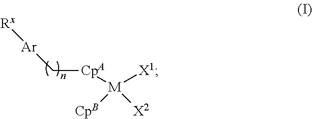
wherein:
M is Zr, Ti, or Hf;
X¹ and X² independently are a monoanionic ligand;
Cp^{A} is a cyclopentadienyl, indenyl, or fluorenyl group with substituent —(CH₂)ₙArR^{x}, and optionally substituted with one or more other substituents;
Cp^{B} is a substituted or unsubstituted cyclopentadienyl, indenyl, or fluorenyl group;
Ar is an aryl group comprising a halogen substituent;
R^{x} is a C₁ to C₁₈ hydrocarbyl group substituent on Ar; and
n is an integer from 0 to 5.

10. The metallocene compound of claim 9, wherein X¹ and X² independently are H, F, Cl, Br, or a C₁ to C₁₂ hydrocarbyl group.

11. The metallocene compound of claim 9, wherein Ar is a phenyl group with two, three, or four halogen substituents.

12. The metallocene compound of claim 11, wherein each halogen substituent is F.

13. The metallocene compound of claim 9, wherein Ar is selected from: 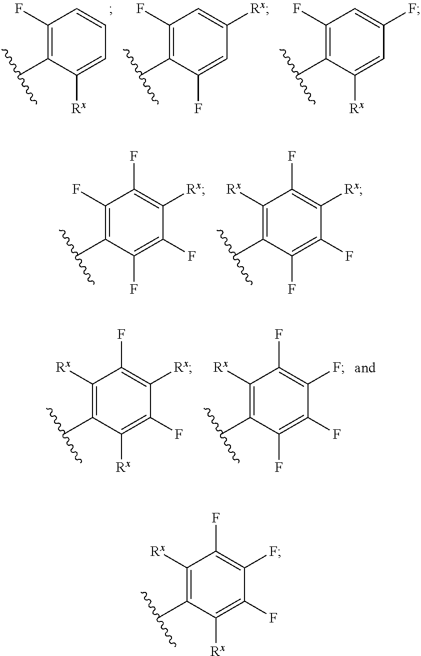
and
R^{x} is selected from methyl, ethyl, n-propyl, n-butyl, sec-butyl, t-butyl, 3-butenyl, n-hexyl, phenyl, and substituted phenyl.

14. The metallocene compound of claim 9, wherein the metallocene compound is selected from:

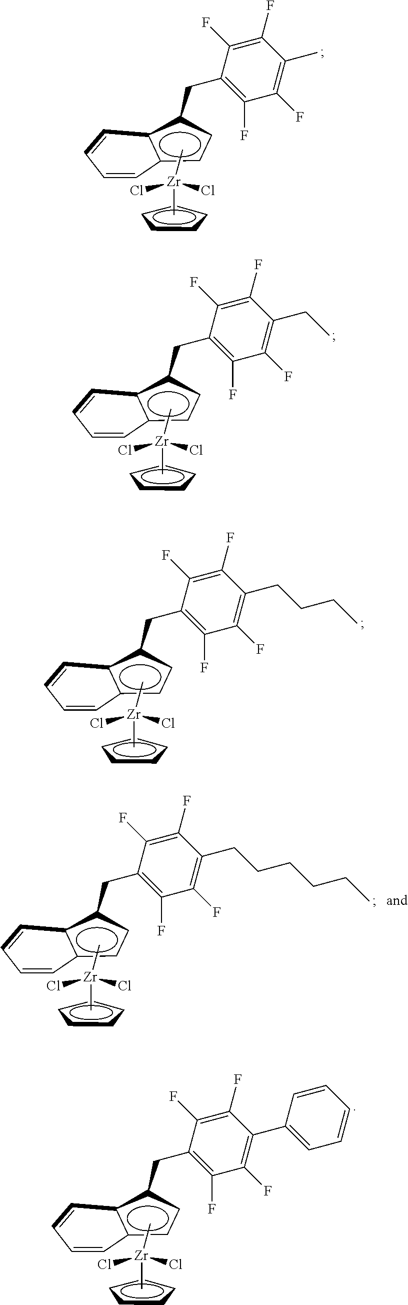

15. The metallocene compound of claim 9, wherein the metallocene compound has a solubility at 25° C. in 1-decene in a range from 0.1 wt. % to 1.0 wt. %.

16. The metallocene compound of claim 9, wherein the metallocene compound has a solubility at 25° C. in 1-decene in a range from 0.2 wt. % to 0.5 wt. %.

17. A catalyst composition comprising:
the metallocene compound of claim 9;
an activator comprising an aluminoxane compound, an organoboron or organoborate compound, an ionizing ionic compound, a chemically treated solid oxide, or any combination thereof; and
optionally, a co-catalyst.

18. The catalyst composition of claim 17, wherein:
the activator comprises the chemically treated solid oxide; and
the co-catalyst comprises triisobutylaluminum.

19. An oligomerization process comprising contacting the catalyst composition of claim 17 with an alpha olefin monomer and optionally H₂ under oligomerization conditions to produce an oligomer product.

20. A polymerization process, the process comprising contacting the catalyst composition of claim 17 with an ethylene monomer and an optional α-olefin comonomer in a polymerization reactor system under polymerization conditions to produce an ethylene polymer.
